SEQUENCE LISTING (47 sequences)

SEQ ID NO: 1 - protein, 661 aa, Artificial Sequence
  FIT-Ig long chain
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 222 aa, Artificial Sequence
  FIT-Ig short chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 214 aa, Artificial Sequence
  FIT-Ig short chain
EIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 684 aa, Artificial Sequence
  FIT-Ig1 heavy chain with leader
MDMRVPAQLLGLLLLWFPGSRCEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 22 aa, Artificial Sequence
  isolated leader sequence
MDMRVPAQLLGLLLLWFPGSRC

SEQ ID NO: 6 - protein, 213 aa, Artificial Sequence
  VL-CL portion of FIT-Ig1 chain 1
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 222 aa, Artificial Sequence; identical to SEQ ID NO: 2
  VH-CH1 portion of FIT-Ig1 heavy chain

SEQ ID NO: 8 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 245 aa, Artificial Sequence
  light chain 1 of FIT-Ig1, with leader
MEFGLSWLFLVAILKGVQCQVQLVQSGAEVKKPGSSVKVSCKTSGDTFSSYAISWVRQAPGQGLEWMGGIIPIFGRAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 10 - protein, 19 aa, artificial sequence
  isolated leader sequence
MEFGLSWLFLVAILKGVQC

SEQ ID NO: 11 - protein, 226 aa, Artificial Sequence
  light chain 1 of FIT-Ig1
QVQLVQSGAEVKKPGSSVKVSCKTSGDTFSSYAISWVRQAPGQGLEWMGGIIPIFGRAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 12 - protein, 236 aa, Artificial Sequence
  light chain 2 of FIT-Ig1
MDMRVPAQLLGLLLLWFPGSRCDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 214 aa, artificial sequence
  light chain 2 of FIT-Ig1
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 689 aa, artificial sequence
  heavy chain of FIT-Ig2
MDMRVPAQLLGLLLLWFPGSRCDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGAEVKKPGSSVKVSCKTSGDTFSSYAISWVRQAPGQGLEWMGGIIPIFGRAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, Artificial Sequence; identical to SEQ ID NO: 13
  VL-CL portion of FIT-Ig2

SEQ ID NO: 16 - protein, 226 aa, Artificial Sequence; identical to SEQ ID NO: 11
  VH(1B12)-CH1 portion of FIT-Ig2

SEQ ID NO: 17 - protein, 241 aa, Artificial Sequence
  first light chain of FIT-Ig2
MEFGLSWLFLVAILKGVQCQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 222 aa, Artificial Sequence; identical to SEQ ID NO: 2
  VH(panitumumab)-CH1 portion of FIT-Ig2

SEQ ID NO: 19 - protein, 235 aa, Artificial Sequence
  second light chain of FIT-Ig2
MDMRVPAQLLGLLLLWFPGSRCEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 213 aa, Artificial Sequence; identical to SEQ ID NO: 6
  VL(1B12)-CL portion of FIT-Ig2

SEQ ID NO: 21 - protein, 685 aa, Artificial Sequence
  heavy chain of FIT-Ig3, with leader sequence
MDMRVPAQLLGLLLLWFPGSRCDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, Artificial Sequence
  VL(10A5)-CL portion of FIT-Ig3
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 222 aa, Artificial Sequence; identical to SEQ ID NO: 2
  VH(panitumumab)-CH1 portion of FIT-Ig heavy chain

SEQ ID NO: 24 - protein, 240 aa, Artificial Sequence
  first light chain of FIT-Ig3
MEFGLSWLFLVAILKGVQCQVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 25 - protein, 221 aa, Artificial Sequence
  VH(10A5)-CH1 portion of FIT-Ig3
QVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 236 aa, Artificial Sequence; identical to SEQ ID NO: 12
  second light chain of FIT-Ig3

SEQ ID NO: 27 - protein, 214 aa, Artificial Sequence; identical to SEQ ID NO: 13
  VL(panitumumab)-CL portion of FIT-Ig3

SEQ ID NO: 28 - protein, 684 aa, Artificial Sequence
  heavy chain of FIT-Ig4, with leader sequence
MDMRVPAQLLGLLLLWFPGSRCDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 214 aa, Artificial Sequence; identical to SEQ ID NO: 13
  VL(panitumumab)-CL portion of FIT-Ig4

SEQ ID NO: 30 - protein, 221 aa, Artificial Sequence; identical to SEQ ID NO: 25
  VH(10A5)-CH1 portion of FIT-Ig4

SEQ ID NO: 31 - protein, 241 aa, Artificial Sequence; identical to SEQ ID NO: 17
  first light chain of FIT-Ig4, with leader sequence

SEQ ID NO: 32 - protein, 222 aa, Artificial Sequence; identical to SEQ ID NO: 2
  VH(panitumumab)-CH1 portion of FIT-Ig4

SEQ ID NO: 33 - protein, 236 aa, Artificial Sequence
  second light chain of FIT-Ig4, with leader sequence
MDMRVPAQLLGLLLLWFPGSRCDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 214 aa, Artificial Sequence; identical to SEQ ID NO: 22
  VL(10A5)-CL portion of FIT-Ig4

SEQ ID NO: 35 - protein, 685 aa, Artificial Sequence
  heavy chain of FIT-Ig5, with leader sequence
MDMRVPAQLLGLLLLWFPGSRCEIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 663 aa, Artificial Sequence
  heavy chain of FIT-Ig5
EIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 214 aa, Artificial Sequence; identical to SEQ ID NO: 3
  VL(3G10)-CL portion of FIT-Ig5

SEQ ID NO: 38 - protein, 222 aa, Artificial Sequence; identical to SEQ ID NO: 2
  VH(panitumumab)-CH1 portion of FIT-Ig5

SEQ ID NO: 39 - protein, 239 aa, Artificial Sequence
  first light chain of FIT-Ig5, with leader sequence
MEFGLSWLFLVAILKGVQCQVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 40 - protein, 220 aa, Artificial Sequence
  VH(3G10)-CH1 portion of FIT-Ig5
QVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 41 - protein, 236 aa, Artificial Sequence; identical to SEQ ID NO: 12
  second light chain of FIT-Ig5, with leader sequence

SEQ ID NO: 42 - protein, 214 aa, Artificial Sequence; identical to SEQ ID NO: 13
  second light chain of FIT-Ig5

SEQ ID NO: 43 - protein, 683 aa, Artificial Sequence
  heavy chain of FIT-Ig6, with leader sequence
MDMRVPAQLLGLLLLWFPGSRCDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 214 aa, Artificial Sequence; identical to SEQ ID NO: 13
  VL(panitumumab)-CL portion of FIT-Ig6 heavy chain

SEQ ID NO: 45 - protein, 220 aa, Artificial Sequence; identical to SEQ ID NO: 40
  VH(3G10)-CH1 portion of FIT-Ig6 heavy chain

SEQ ID NO: 46 - protein, 241 aa, Artificial Sequence; identical to SEQ ID NO: 17
  first light chain of FIT-Ig6, with leader sequence

SEQ ID NO: 47 - protein, 236 aa, Artificial Sequence
  second light chain of FIT-Ig6, with leader sequence
MDMRVPAQLLGLLLLWFPGSRCEIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0017617 A1
(43) Pub. Date: Jan. 20, 2022

(54) EFFICIENTLY EXPRESSED EGFR AND PD-L1 BISPECIFIC BINDING PROTEINS

(71) Applicant: SHANGHAI EPIMAB BIOTHERAPEUTICS CO., LTD., Shanghai (CN)

(72) Inventors: Chengbin WU, Shanghai (CN); Shiyong GONG, Shanghai (CN)

(73) Assignee: SHANGHAI EPIMAB BIOTHERAPEUTICS CO., LTD., Shanghai (CN)

(21) Appl. No.: 17/250,353

(22) PCT Filed: Jul. 8, 2019
(86) PCT No.: PCT/US19/40762

§ 371 Date: Jan. 8, 2021

(30) Foreign Application Priority Data
Jul. 9, 2018 (CN) PCT/CN2018/095000

(51) Int. Cl. C07K 16/28; C12N 15/86; C07K 16/46; C07K 16/30

(52) U.S. Cl. CPC C07K 16/2818; C12N 15/86; C07K 16/468; C07K 2317/515; C07K 16/30; C07K 2317/732; C07K 2317/92; C07K 16/2863

(57) ABSTRACT

Bispecific Fabs-In-Tandem Immunoglobulin (FTT-Ig) binding proteins that bind both EGFR and PD-L1 simultaneously are disclosed. Such bispecific EGFR/PD-L1 FIT-Ig binding proteins are efficiently expressed and are useful for blocking EGFR signaling, for blocking PD-L1 signaling, and for treating cancer.

### RELATED APPLICATIONS

This application is a U.S. national stage entry under 35 U.S.C. § 371 of international application number PCT/US2019/040762, filed Jul. 8, 2019, which designates the U.S. and claims priority to International Application No. PCT/CN2018/09500, filed Jul. 9, 2018, each of which is incorporated herein by reference in its entirety.

### FIELD OF THE INVENTION

The present invention relates to new engineered bispecific binding proteins recognizing epidermal growth factor receptor (EGFR) and Programmed Death Ligand 1 (PD-L1). The bispecific binding proteins are useful in the treatment of cancer.

### BACKGROUND OF THE INVENTION

Programmed Death Ligand 1 (PD-L1) is a type I transmembrane glycoprotein of about 40 kilodaltons (kD) in size. In humans, PD-L1 is expressed on a number of immune cell types including activated and anergic/exhausted T cells, naive and activated B cells, myeloid dendritic cells (DCs), monocytes, mast cells, and other antigen presenting cells (APCs). It is also expressed on non-immune cells including islets of the pancreas, Kupffer cells of the liver, vascular endothelium, and selected epithelia, for example, airway epithelia and renal tubule epithelia, where its expression is enhanced during inflammatory episodes. PD-L1 expression is also found at increased levels on a number of malignant tumors including, but not limited to, breast cancer, colon cancer, colorectal cancer, lung cancer, renal cancer (including renal cell carcinoma), gastric cancer, bladder cancer, non-small cell lung cancer (NSCLC), hepatocellular cancer (HCC), pancreatic cancer, and melanoma. Expression of PD-L1 on the cell surface has also been shown to be upregulated through IFN-γ (gamma interferon) stimulation.

PD-L1 (CD274, B7-H1) binds Programmed Cell Death Protein 1 (PD-1, CD279), which is a member of the CD28 family of receptors, which includes CD28, CTLA-4, ICOS, PD-1, and BTLA. Expression of PD-1 is frequently found in immune cells such as T cells, B cells, monocytes, and natural killer (NK) cells. PD-L1 and also PD-L2 (CD273, B7-DC) are both cell surface glycoprotein ligands for PD-1. The binding of PD-1 to PD-L1 or PD-L2 signals an inhibition of T cell activation and cytokine secretion. This downregulation of T cell activation in turn results in reduction of T cell proliferation, IL-2 secretion, IFN-γ secretion, and secretion of other growth factors and cytokines. Freeman et al., J. Exp. Med., 192: 1027-1034 (2000); Latchman et al., Nat. Immunol., 2: 261-8 (2001); Carter et al., Eur. J. Immunol., 32: 634-43 (2002); Ohigashi et al., Clin. Cancer Res., 11: 2947-53 (2005). Signaling via the PD-1/PD-L1 interaction is believed to serve critical, non-redundant functions within the immune system, by negatively regulating T cell responses. This regulation is involved in T cell development in the thymus, in regulation of chronic inflammatory responses, and in maintenance of both peripheral tolerance and immune privilege. The critical nature of these functions is exemplified in PD-1-deficient mice, which exhibit an autoimmune phenotype. PD-1 deficiency in C57BL/6 mice results in chronic progressive lupus-like glomerulonephritis and arthritis. In Balb/c mice, PD-1 deficiency leads to severe cardiomyopathy due to the presence of heart-tissue-specific self-reacting antibodies.

PD-L1 has been suggested to play a role in tumor immunity by increasing apoptosis of antigen-specific T-cell clones. Dong et al., Nat. Med., 8:793-800 (2002). It has also been suggested that PD-L1 might be involved in intestinal mucosal inflammation, and inhibition of PD-L1 suppresses wasting disease associated with colitis. Kanai et al., J. Immunol., 171: 4156-63 (2003). In general, the inhibition of PD-L1 signaling has been proposed as a means to enhance T cell immunity for the treatment of cancer (e.g., tumor immunity) and infection, including both acute and chronic (e.g., persistent) infection.

Owing to their involvement in down-regulating the immune response, including the suppression of anti-tumor immune responses, PD-L1, PD-L2, and PD-1 are known as “immune checkpoint” proteins. Pardoll, Nat. Rev. Cancer, 12: 252-264 (2012). Clinical studies using immune checkpoint inhibitors, such as antibodies targeting PD-1, PD-L1, or CTLA-4, have led to promising results, however, it has been observed that only a subset of patients initially respond to current inhibitors, and increasing clinical evidence indicates that a substantial proportion of initial responders ultimately relapse, with lethal, drug-resistant disease months or years later. Syn et al., The Lancet Oncology, 18(12): e731-e741 (2017).

Epidermal growth factor receptor (EGFR) is a transmembrane glycoprotein and member of the ErbB superfamily of receptor tyrosine kinases. EGFR has been shown to play a major role in a complex signaling cascade that promotes development, survival, and metastasis of epithelial cancers. EGFR signaling is triggered when EGFR binds its cognate ligand epidermal growth factor (EGF) and then forms dimers, either with another EGFR (homodimerization) or another receptor tyrosine kinase (heterodimerization). Thereafter, the dimer is internalized for degradation or accumulation in the cell nucleus where the EGFR can regulate transcription of various genes involved in cancer transformation. Thus, EGFR has been recognized as an attractive therapeutic target for anti-tumor therapies. Approved anti-cancer therapies targeting EGFR include the monoclonal antibodies cetuximab, which is a human-murine chimeric anti-EGFR monoclonal antibody, and panitumumab, which is a human anti-EGFR monoclonal antibody. A number of small molecule tyrosine kinase inhibitors that inhibit EGFR and other receptor tyrosine kinases have been approved as anti-cancer therapies including gefitinib, erlotinib, lapatinib, and canertinib. These approved drugs have been used alone or in various combinations to treat various cancers. For a review on targeting EGFR in anti-cancer therapies, see Seshacharyulu et al., Expert Opin. Ther. Targets, 16(1): 15-31 (2012).

PD-L1 and EGFR are involved in the regulation of different signaling pathways, both of which are known to contribute to the initiation, growth, maintenance, and spread of cancer cells in the human body. However, in some cancer cells, activation of EGFR has been shown to up-regulate PD-L1 expression, indicating some degree of “cross-talk” between the two pathways (Chen et al., J. Thorac. Oncol., 10(6): 910-923 (2015). A therapy that inhibits both of these proteins to block their respective regulatory functions may offer an effective approach for treating a variety of cancers.

### SUMMARY OF THE INVENTION

This invention addresses the above needs by providing engineered bispecific proteins that bind both EGFR and PD-L1. In particular, the invention provides bispecific, multivalent binding proteins that bind human EGFR and human PD-L1. A preferred bispecific binding protein of the invention is a “Fabs-In-Tandem Immunoglobulin” (FIT-Ig) binding protein that binds both EGFR and PD-L1. As shown herein, such an “EGFR/PD-L1” FIT-Ig binding protein according to the invention is produced in significantly high yields in mammalian cell cultures and exhibits no significant aggregate formation. Low production yields and significant aggregate formation are problems that have rendered previously made FIT-Ig binding proteins directed to EGFR and PD-L1 impractical for conducting the pre-clinical and clinical stage assessments that are needed to determine whether such binding proteins may be used as therapeutic anti-cancer drugs.

In an embodiment, the invention provides an EGFR/PD-L1 FIT-Ig binding protein that binds EGFR and PD-L1 and that comprises a first polypeptide chain, a second polypeptide chain, and a third polypeptide chain, wherein:

the first polypeptide chain (“heavy chain”) comprises, from the amino terminus to the carboxy terminus, VL_{EGFR}-CL-VH_{PD-L1}-CH1-Fc, wherein VL_{EGFR} is an antibody light chain variable domain of a first parental antibody that binds EGFR, CL is an antibody light chain constant domain, VH_{PD-L1} is an antibody heavy chain variable domain of a second parental antibody that binds PD-L1, CH1 is a first constant domain of an antibody heavy chain, Fc is an antibody Fc region (comprising hinge-CH2-CH3); wherein CL is fused directly to VH_{PD-L1}, wherein there is no artificial linker inserted between variable and constant domains, and wherein:
VL_{EGFR} comprises amino acid residues 1-107 of SEQ ID NO:1 and
VH_{PD-L1} comprises amino acid residues 215-331 of SEQ ID NO:1;

the second polypeptide chain (“first light chain”) comprises, from the amino terminus to carboxy terminus, VH_{EGFR}-CH1, wherein VH_{EGFR} is an antibody heavy chain variable domain of said first parental antibody that binds EGFR, wherein CH1 is a first constant domain of an antibody heavy chain, wherein there is no artificial linker inserted between VH_{EGFR} and CH1, and wherein:
VH_{EGFR} comprises amino acid residues 1-119 of SEQ ID NO:2;

the third polypeptide chain (“second light chain) comprises, from the amino terminus to the carboxy terminus, VL_{PD-L1}-CL, wherein VL_{PD-L1} is a light chain variable domain of said second parental antibody that binds PD-L1, wherein CL is an antibody light chain constant domain, wherein there is no artificial linker inserted between VL_{PD-L1} and CL. and wherein:
VL_{PD-L1} comprises amino acid residues 1-107 of SEQ ID NO:3.

In a preferred embodiment, the EGFR/PD-L1 FIT-Ig binding protein described above is a six-polypeptide chain FIT-Ig binding protein that comprises two of the first polypeptide chains described above, two of the second polypeptide chains described above, and two of the third polypeptide chains described above, wherein the polypeptide chains associate to form four Fab binding units, wherein two of the Fab binding units bind EGFR and two of the Fab binding units bind PD-L1.

Preferably, a CL domain present in one or more polypeptide chains, such as the first polypeptide chain and the third polypeptide chain described above, of a FIT-Ig binding protein of the invention is a human CL kappa domain (hCκ). Preferably, a CL domain of a FIT-Ig binding protein of the invention is derived from a human IgG1 (hIgG1) antibody. A preferred hIgG1 CL kappa domain present in one or more polypeptide chains of an EGFR/PD-L1 FIT-Ig binding protein of the invention comprises amino acid residues 108-214 of SEQ ID NO:1.

Preferably, a CH1 domain present in one or more polypeptide chains, such as the first polypeptide chain and the second polypeptide chain described above, of a FIT-Ig binding protein of the invention is derived from a human IgG1 antibody. A preferred hIgG1 CH1 domain present in one or more polypeptide chains of an EGFR/PD-L1 FIT-Ig binding protein of the invention comprises amino acid residues 332-434 of SEQ ID NO:1.

Preferably, an Fc in a polypeptide chain that is present in the first polypeptide chain (or “heavy chain”), such as described above, of a FIT-Ig binding protein of the invention comprises an antibody Fc region comprising hinge-CH2-CH3 domains. Preferably, the Fc is derived from a human IgG1 antibody. A preferred hIgG1 Fc region present in a first polypeptide chain of an EGFR/PD-L1 FIT-Ig binding protein of the invention comprises amino acid residues 435-661 of SEQ ID NO:1.

The invention also provides an EGFR/PD-L1 FIT-Ig binding protein that binds EGFR and PD-L1 and that comprises:

a first polypeptide chain that comprises a sequence of amino acid residues according to SEQ ID NO:1;

a second polypeptide chain that comprises a sequence of amino acid residues according to SEQ ID NO:2; and

a third polypeptide chain that comprises a sequence of amino acid residues according to SEQ ID NO:3;

wherein the EGFR/PD-L1 FIT-Ig comprises a Fab binding unit for EGFR and a Fab binding unit for PD-L1.

In a preferred embodiment, the EGFR/PD-L1 FIT-Ig binding protein described above is a six-polypeptide chain FIT-Ig binding protein that comprises two of the first polypeptide chains that comprise a sequence of amino acid residues according to SEQ ID NO:1, two of the second polypeptide chains that comprise a sequence of amino acid residues according to SEQ ID NO:2, and two of the third polypeptide chains that comprise a sequence of amino acid residues according to SEQ ID NO:3, and wherein the polypeptide chains associate to form four Fab binding units, wherein two of the Fab binding units bind EGFR and two of the Fab binding units bind PD-L1.

Preferably, an EGFR/PD-L1 FIT-Ig binding protein described herein binds EGFR and PD-L1 simultaneously. In another embodiment, an EGFR/PD-L1 FIT-Ig binding protein of the invention binds two EGFR proteins and two PD-L1 proteins. In a more preferred embodiment, an EGFR/PD-L1 FIT-Ig binding protein described herein binds two EGFR proteins and two PD-L1 proteins simultaneously.

In an embodiment, an EGFR/PD-L1 FIT-Ig binding protein according to the invention binds EGFR and PD-L1, wherein the affinities for EGFR and PD-L1 are substantially the same as (i.e., same as or within 30% of) the affinities for EGFR and for PD-L1 of the respective parental antibodies from which the individual EGFR and PD-L1 antigen-binding sites of the FIT-Ig binding protein are derived.

In an embodiment, an EGFR/PD-L1 FIT-Ig binding protein of the invention binds EGFR and has an on-rate constant (kₒₙ) for human EGFR that is at least 1×10⁵ M⁻¹s⁻¹, more preferably at least 2×10⁵ M⁻¹s⁻¹, as determined by biolayer interferometry. In a further embodiment, an EGFR/PD-L1 FIT-Ig binding protein according to this invention has a kₒₙ for human EGFR that is approximately 40% lower than the kₒₙ for human EGFR of the parental antibody from which the anti-EGFR specificity of the EGFR/PD-L1 FTI-Ig binding protein was derived.

In an embodiment, an EGFR/PD-L1 FIT-Ig binding protein of the invention binds human EGFR and has an off-rate constant (k_{off}) for human EGFR that is less than 1.1×10⁻⁴ sec⁻¹, as determined by biolayer interferometry. In a further embodiment, an EGFR/PD-L1 FIT-Ig binding protein of the invention has a k_{off} for human EGFR that is approximately 50% lower than the value of the k_{off} for human EGFR of the parental antibody from which the anti-EGFR specificity of the EGFR/PD-L1 FIT-Ig binding protein was derived.

In an embodiment, an EGFR/PD-L1 FIT-Ig binding protein of the invention binds human EGFR and has a dissociation constant (K_{D}) for human EGFR that is less than 1×10⁻⁹ M, preferably less than 7×10⁻¹⁰ M, more preferably less than 6×10⁻¹⁰ M, and still more preferably less than or equal to 5×10⁻¹⁰ M as determined by biolayer interferometry. In a further embodiment, an EGFR/PD-L1 FIT-Ig binding protein of the invention has a K_{D} for human EGFR that is substantially the same as (i.e., identical to or within 25% of) the K_{D} for human EGFR of the parental antibody from which the anti-EGFR specificity of the EGFR/PD-L1 FIT-Ig binding protein was derived.

In an embodiment, an EGFR/PD-L1 FIT-Ig binding protein of the invention binds PD-L1 and has an on-rate constant (kₒₙ) for human PD-L1 that is at least 5×10⁵ M⁻¹s⁻¹, more preferably at least 7×10⁵ M⁻¹s⁻¹, and still more preferably at least 8×10⁵ M⁻¹s⁻¹, as determined by biolayer interferometry. In a further embodiment, an EGFR/PD-L1 FIT-Ig binding protein according to this invention has a kₒₙ for human PD-L1 that is the same as or within approximately 90% of the kₒₙ for human PD-L1 of the parental antibody from which the anti-PD-L1 specificity of the EGFR/PD-L1 FTI-Ig binding protein was derived.

In an embodiment, an EGFR/PD-L1 FIT-Ig binding protein of the invention binds human PD-L1 and has an off-rate constant (k_{off}) for human PD-L1 that is less than 2×10⁻² sec⁻¹ and more preferably less than 1.5×10⁻² sec⁻¹ as determined by biolayer interferometry. In a further embodiment, an EGFR/PD-L1 FIT-Ig binding protein of the invention has a k_{off} for human PD-L1 that is approximately 20% higher than k_{off} for human PD-L1 of the parental antibody from which the anti-PD-L1 specificity of the EGFR/PD-L1 FIT-Ig binding protein was derived.

In an embodiment, an EGFR/PD-L1 FIT-Ig binding protein of the invention binds human PD-L1 and has a dissociation constant (K_{D}) for PD-L1 that is less than 2×10⁻⁸ M and more preferably less than 1.7×10⁻⁸ M as determined by biolayer interferometry. In a further embodiment, an EGFR/PD-L1 FIT-Ig binding protein of the invention has a K_{D} for PD-L1 that is substantially the same as (i.e., identical to or within 30% of) the K_{D} for PD-L1 of the parental antibody from which the anti-PD-L1 specificity of the EGFR/PD-L1 FIT-Ig binding protein was derived.

In an embodiment, an EGFR/PD-L1 FIT-Ig binding protein according to the invention is expressed in a mammalian cell culture at a level that is greater than 10 mg/L.

A fully assembled, six-polypeptide chain EGFR/PD-L1 FIT-Ig binding protein “monomer” can be purified from cell culture medium using Protein A affinity chromatography. A solution or suspension of EGFR/PD-L1 FIT-Ig binding protein that has been purified using Protein A affinity chromatography can be further analyzed for possible aggregates using size exclusion chromatography (SEC), wherein protein aggregates are detected as molecular species that have molecular weights greater than that of the six-chain EGFR/PD-L1 FIT-Ig binding protein monomer, which has a molecular weight of approximately 240,000 daltons. In an embodiment, the invention provides a composition (for example, a solution or suspension) comprising an EGFR/PD-L1 FIT-Ig binding protein described herein that has been purified using Protein A affinity chromatography (preferably, column) and that has less than or equal to 0.1% (≤0.1%) FIT-Ig protein aggregates.

In another embodiment, an EGFR/PD-L1 FIT-Ig binding protein described herein is glycosylated. Preferably, the glycosylation is a human glycosylation pattern.

In an embodiment, an EGFR/PD-L1 FIT-Ig binding protein described herein inhibits or blocks EGFR signaling or PD-L1 signaling. Preferably, an EGFR/PD-L1 FIT-Ig binding protein of the invention inhibits or blocks both EGFR signaling and PD-L1 signaling

In an embodiment, an EGFR/PD-L1 FIT-Ig binding protein described herein inhibits the growth or survival of cancer cells.

The invention also provides one or more isolated nucleic acids encoding one or more of the polypeptide chains of an EGFR/PD-L1 FIT-Ig binding protein described above.

In a preferred embodiment, an isolated nucleic acid molecule of the invention encodes a first polypeptide chain (heavy chain), a second polypeptide chain (first light chain), or a third polypeptide chain (second light chain) of an EGFR/PD-L1 FIT-Ig binding protein, wherein:

the first polypeptide chain (heavy chain) comprises an amino acid sequence according to SEQ ID NO:1;

a second polypeptide chain (first light chain) comprises an amino acid sequence according to SEQ ID NO:2; and

a third polypeptide chain (second light chain) comprises an amino acid sequence according to SEQ ID NO:3.

In an embodiment, the invention provides an expression vector comprising one or more isolated nucleic acid molecules described above encoding one or more polypeptide chains of an EGFR/PD-L1 FIT-Ig binding protein, wherein the one or more isolated nucleic acid molecules is operably linked to appropriate transcriptional and/or translational sequences required for expression of the one or more encoded polypeptide chains of an EGFR/PD-L1 FIT-Ig binding protein in a host cell that is compatible with the expression vector. Preferably, a single expression vector comprises a single nucleic acid encoding only one of the three component polypeptide chains of a FIT-Ig binding protein described herein so that three separate expression vectors (each encoding and expressing only of the three component polypeptides) must be present in a host cell to produce a FIT-Ig binding protein described herein.

Preferred expression vectors for cloning and expressing nucleic acids described herein include, but are not limited to, pcDNA, pcDNA3.1, pTT (Durocher et al, Nucleic Acids Res., 30(2e9): 1-9 (2002)), pTT3 (pTT with additional multiple cloning sites), pEFBOS (Mizushima and Nagata, Nucleic Acids Res., 18(17): 5322 (1990)), pBV, pJV, pcDNA3.1 TOPO, pEF6 TOPO, and pBJ.

A vector of the invention may be an autonomously replicating vector or a vector that can incorporate into the genome of a host cell.

In another embodiment, the invention provides an isolated host cell comprising one or more vectors described above. Such an isolated host cell may be an isolated prokaryotic cell or an isolated eukaryotic cell.

In an embodiment of the invention, an isolated prokaryotic host cell comprising one or more vectors described herein is a bacterial host cell. The bacterial host cell may be a Gram positive, Gram negative, or Gram variable bacterial cell. Preferably, the bacterial host cell comprising one or more vectors described herein is a Gram negative bacterium. Even more preferably, a bacterial host cell comprising one or more vectors described herein is an Escherichia coli cell.

In an embodiment of the invention, an isolated host cell comprising one or more vectors described herein is a eukaryotic host cell. Examples of an isolated eukaryotic host cell that may comprise one or more vectors described herein include, without limitation, a mammalian host cell, an insect host cell, a plant host cell, a fungal host cell, a eukaryotic algal host cell, a nematode host cell, a protozoan host cell, and a fish host cell.

An isolated fungal host cell that may comprise one or more vectors described herein is selected from the group consisting of: Aspergillus, Neurospora, Saccharomyces, Pichia, Hansenula, Schizosaccharomyces, Kluyveromyces, Yarrowia, and Candida. A preferred fungal host cell is a Saccharomyces host cell. More preferably, the Saccharomyces host cell is a Saccharomyces cerevisiae cell. An insect cell useful as a host cell according to the invention is an insect Sf9 cell.

In a preferred embodiment, a host cell according to the invention is an isolated mammalian host cell that comprises one or more expression vectors described herein, wherein the mammalian host cell expresses the three polypeptide chains encoded on the one or more expression vectors, and wherein the polypeptide chains associate to form a FIT-Ig binding protein that comprises two Fab binding units that bind EGFR and two Fab binding units that bind PD-L1. Particularly preferred is a mammalian host cell selected from the group consisting of: a Chinese Hamster Ovary (CHO) cell, a COS cell, a Vero cell, an SP2/0 cell, an NS/0 myeloma cell, a human embryonic kidney (HEK293) cell, a baby hamster kidney (BHK) cell, a HeLa cell, a human B cell, a CV-1/EBNA cell, an L cell, a 3T3 cell, an HEPG2 cell, a PerC6 cell, and an MDCK cell.

More preferably, an isolated mammalian host cell according to the invention comprises three expression vectors, wherein each expression vector encodes and expresses one of the three component polypeptide chains of a FIT-Ig binding protein described herein, and wherein the three expressed polypeptide chains associate to form a FIT-Ig binding protein that comprises two Fab binding units that bind EGFR and two Fab binding units that bind PD-L1.

The invention also provides a method of producing an EGFR/PD-L1 FIT-Ig binding protein described herein comprising culturing an isolated host cell comprising one or more expression vectors described herein under conditions sufficient to produce the EGFR/PD-L1 FIT-Ig binding protein.

Another aspect of the invention is an EGFR/PD-L1 FIT-Ig binding protein produced by a method comprising culturing an isolated host cell comprising one or more expression vectors described herein under conditions sufficient to produce the EGFR/PD-L1 FIT-Ig binding protein.

An EGFR/PD-L1 FIT-Ig binding protein described herein may be conjugated to another compound, for example, along or at the carboxy terminus of the CH3 domain of the Fc region of one or both first (heavy) polypeptide chains in a manner similar to that of other conjugated antibodies. Such compounds that may be conjugated to an EGFR/PD-L1 FIT-Ig binding protein include, but are not limited to, an imaging agent and therapeutic agents. Preferred imaging agents that may be conjugated to an EGFR/PD-L1 FIT-Ig binding protein include, without limitation, a radiolabel, an enzyme, a fluorescent label, a luminescent label, a bioluminescent label, a magnetic label, biotin, streptavidin, and avidin. Radiolabels that may be conjugated to an EGFR/PD-L1 FIT-Ig binding protein described herein include, but are not limited to, ³H, ¹⁴C, ³⁵S, ⁹⁰Y, ⁹⁹Tc, ¹¹¹In, ¹³¹I, ¹⁷⁷Lu, ¹⁶⁶Ho, and ¹⁵³ Sm. Preferred therapeutic compounds that may be conjugated to an EGFR/PD-L1 FIT-Ig binding protein described herein include, but are not limited to, an antibiotic, an anti-viral agent, a small molecule receptor tyrosine kinase inhibitor, and a cytokine.

In another embodiment, an EGFR/PD-L1 FIT-Ig binding protein described herein may be a crystallized EGFR/PD-L1 FIT-Ig binding protein that retains the binding affinity for EGFR and PD-L1 of the non-crystallized EGFR/PD-L1 FIT-Ig binding protein. Such a crystallized EGFR/PD-L1 FIT-Ig binding protein may also provide carrier-free controlled release of the EGFR/PD-L1 FIT-Ig binding protein when administered to an individual. A crystallized EGFR/PD-L1 FIT-Ig binding protein of the invention may also exhibit a greater in vivo half-life when administered to an individual compared to the non-crystallized form. Crystallized binding protein of the invention may be produced according methods known in the art and as disclosed in International Publication No. WO 02/072636 (Shenoy et al.), incorporated herein by reference.

An embodiment of the invention provides a composition for the release of a crystallized EGFR/PD-L1 FIT-Ig binding protein wherein the composition comprises a crystallized EGFR/PD-L1 FIT-Ig binding protein as described herein, an excipient ingredient, and at least one polymeric carrier. Preferably the excipient ingredient is selected from the group consisting of: albumin, sucrose, trehalose, lactitol, gelatin, hydroxypropyl-β-cyclodextrin, methoxypolyethylene glycol and polyethylene glycol. Preferably the polymeric carrier is a polymer selected from one or more of the group consisting of: poly(acrylic acid), poly(cyanoacrylates), poly(amino acids), poly(anhydrides), poly(depsipeptide), poly(esters), polylactic acid), polylactic-co-glycolic acid) or PLGA, poly(b-hydroxybutyrate), poly(caprolactone), poly(dioxanone); poly(ethylene glycol), poly((hydroxypropyl) methacrylamide, poly[(organo)phosphazene], poly(ortho esters), poly(vinyl alcohol), poly(vinylpyrrolidone), maleic anhydride/alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends thereof, and copolymers thereof.

A pharmaceutical composition of the invention comprises an EGFR/PD-L1 FIT-Ig binding protein described herein and one or more pharmaceutically acceptable components such as a pharmaceutically acceptable carrier (vehicle, buffer), pharmaceutically acceptable excipient, and/or other pharmaceutically acceptable ingredient.

Preferred pharmaceutically acceptable carriers useful in a pharmaceutical composition of the invention include, but are not limited to: water, saline, phosphate buffered saline, dextrose, glycerol, ethanol, and combinations thereof.

A pharmaceutical composition of the invention may further comprise an isotonic agent. A preferred isotonic agent useful in a pharmaceutical composition of the invention is selected from the group consisting of: a sugar, a polyalcohol (such as mannitol or sorbitol), sodium chloride, and combinations thereof.

A pharmaceutical composition comprising an EGFR/PD-L1 FTI-Ig binding protein described herein may further comprise one or more other therapeutically active compounds (therapeutic agents). Examples of such additional therapeutic agents that may be incorporated into a pharmaceutical composition of the invention include, but are not limited to, an anti-cancer agent that is different from an EGFR/PD-L1 FTI-Ig binding protein described herein (for example, a cytotoxic metal-containing anti-cancer compound or a cytotoxic radioisotope-based anti-cancer compound, and combinations thereof), an antibiotic, an anti-viral compound, a sedative, a stimulant, a local anesthetic, an anti-inflammatory steroid (for example, natural or synthetic anti-inflammatory steroids and combinations thereof), an analgesic (for example, acetylsalicylic acid, acetaminophen, naproxen, ibuprofen, a COX-2 inhibitor, morphine, oxycodone, and combinations thereof), an anti-histamine, a non-steroidal anti-inflammatory drug (“NSAID,” for example, acetylsalicylic acid, ibuprofen, naproxen, a COX-2 inhibitor, and combinations thereof), and combinations thereof.

In another embodiment, a pharmaceutical composition of the invention comprises an EGFR/PD-L1 FIT-Ig binding protein as described herein, a pharmaceutically acceptable carrier, and an adjuvant, wherein the adjuvant provides a general stimulation of the immune system of a patient.

In an embodiment, the present invention provides a method for treating cancer in a subject in need thereof, the method comprising administering to the subject an EGFR/PD-L1 FIT-Ig binding protein as described herein.

The invention also provides a method of inhibiting or blocking EGFR signaling in a cell comprising contacting a cell that expresses EGFR with an EGFR/PD-L1 FIT-Ig binding protein described herein.

In another embodiment, the invention provides a method of inhibiting or blocking PD-L1 signaling in a cell comprising contacting a cell that expresses PD-L1 with an EGFR/PD-L1 FIT-Ig binding protein described herein.

In an embodiment, the present invention provides a method for treating cancer in a subject in need thereof, the method comprising administering to the subject a pharmaceutical composition comprising an EGFR/PD-L1 FIT-Ig binding protein as described herein.

In another embodiment, the present invention provides a method for treating cancer in a cancer patient, wherein an EGFR/PD-L1 FIT-Ig binding protein as described herein is administered to the subject, and wherein the cancer is a cancer typically responsive to immunotherapy. In another embodiment, the cancer is a cancer that has not been associated with immunotherapy. In another embodiment, the cancer is a cancer that is a refractory or a recurring malignancy.

Preferably, a cancer that is treated using a method according to the invention is an epithelial cancer.

In another embodiment, a cancer that is treated using a method according to the invention is selected from the group consisting of: a melanoma (for example, metastatic malignant melanoma), renal cancer (e.g. clear cell renal cell carcinoma, “CCRCC”), prostate cancer (for example, hormone refractory prostate adenocarcinoma), pancreatic adenocarcinoma, breast cancer, colon cancer, lung cancer (for example, non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

In another embodiment, the invention provides a method for treating a human subject suffering from a disease in which EGFR and/or PD-L1 activity is detrimental, such method comprising administering to the subject an EGFR/PD-L1 FIT-Ig binding protein of the invention such that an activity mediated by PD-L1/PD1 binding and/or EGFR/EGF binding in the subject is reduced or blocked.

In an embodiment, the invention provides a method of detecting EGFR and/or PD-L1 in a sample, wherein the sample contains or is suspected of containing EGFR or PD-L1 or cells expressing EGFR or PD-L1, wherein the sample is contacted with a FIT-Ig binding protein described herein. For example, an EGFR/PD-L1 FIT-Ig binding protein of the invention can be used to detect EGFR or PD-L1, or both, in a conventional immunoassay, such as an enzyme linked immunosorbent assays (ELISA), a radioimmunoassay (RIA), or tissue immunohistochemistry, wherein the FIT-Ig binding protein is used instead of an anti-EGFR antibody or an anti-PD-L1 antibody. The invention provides a method for detecting EGFR or PD-L1 in a biological sample comprising contacting a biological sample with the EGFR/PD-L1 FIT-Ig binding protein of the invention and detecting whether binding to a target antigen (EGFR or PD-L1) occurs, thereby detecting the presence or absence of the target in the biological sample. The FIT-Ig binding protein may be directly or indirectly labeled with a detectable substance to facilitate detection of the bound or unbound FIT-Ig binding protein. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³³I, ¹⁷⁷Lu, ¹⁶⁶Ho, or ¹⁵³Sm.

### BRIEF DESCRIPTION OF THE DRAWINGS

FIG. 1 is a diagram that illustrates the general procedure for constructing three expression vectors used for expressing the three types of polypeptide chains for each of the FIT-Ig binding proteins described in Example 1.

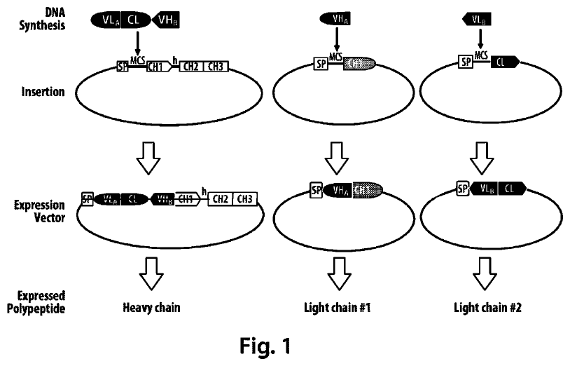

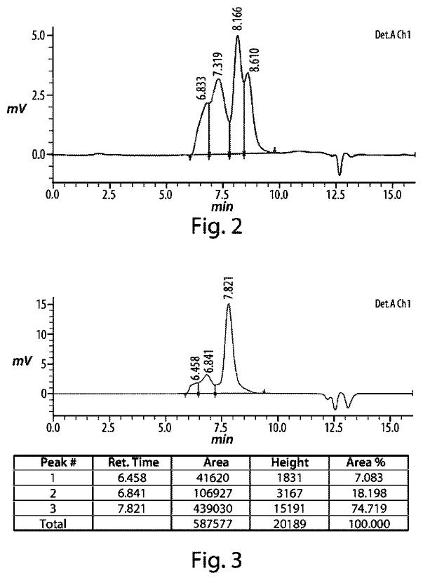

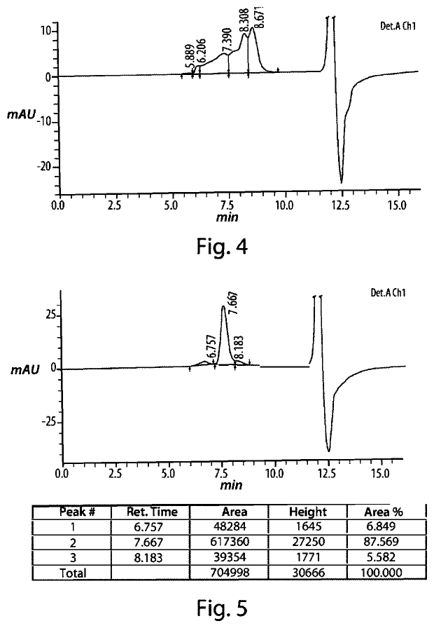

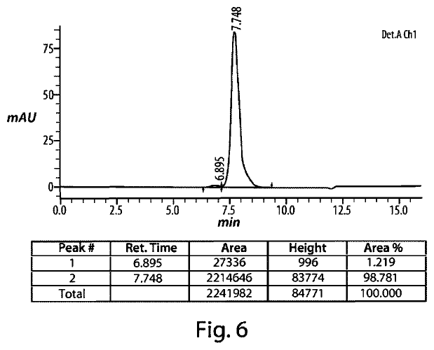

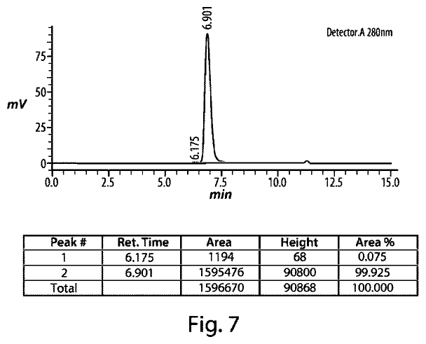

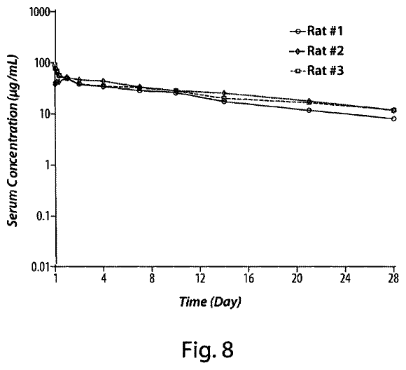

As illustrated in FIG. 1, to express the first polypeptide chain (“Heavy chain”) of a FIT-Ig binding protein, a DNA molecule was synthesized (“DNA Synthesis”) that encoded the VL_{A}-CL-VH_{B} segment of the first polypeptide chain. The DNA molecule was then inserted (“Insertion”) into the multiple cloning site (MCS) of a pcDNA3.1 expression vector so that the inserted DNA molecule was positioned downstream of the vector's strong cytomegalovirus (CMV)-enhancer promoter, also downstream of and in frame with a DNA segment encoding an amino terminal signal peptide (SP), and upstream of and in frame with an inserted DNA molecule encoding an antibody CH1 domain linked to an antibody Fc region comprising the naturally contiguous hinge-CH2-CH3 domains (designated “h-CH2-CH3”).

As illustrated in FIG. 1, to express the second polypeptide chain (“Light chain #1) of a FIT-Ig binding protein, a DNA segment was synthesized that encoded an antibody VH_{A} domain, which was then inserted into the multiple cloning site (MCS) of a pcDNA3.1 expression vector so that the inserted DNA molecule was positioned downstream of the vector's strong CMV-enhancer promoter, also downstream of and in frame with a DNA segment encoding an amino terminal signal peptide (SP), and upstream of and in frame with an inserted DNA molecule encoding an antibody CH1 domain.

To express the third polypeptide chain (“Light chain #2), a DNA segment was synthesized that encoded an antibody VL_{B} domain, which was then inserted into the multiple cloning site (MCS) of a pcDNA3.1 expression vector so that the inserted DNA molecule was positioned downstream of the vector's strong CMV-enhancer promoter, also downstream of and in frame with a DNA segment encoding an amino terminal signal peptide (SP), and upstream of and in frame with an inserted DNA molecule encoding an antibody CL domain.

See, Example 1 for additional details.

FIG. 2 shows a size exclusion chromatography elution profile for a sample of FIT-Ig1 that had previously been purified by Protein A affinity chromatography. The elution profile is complex and indicates a significant proportion of FIT-Ig1 aggregates. The FIT-Ig1 six-chain monomer constituted less than 30% of the purified protein. See, Example 1.7 for details.

FIG. 3 shows a size exclusion chromatography elution profile for a sample of FIT-Ig2 that had previously been purified by Protein A affinity chromatography. The elution profile shows a major peak of the FIT-Ig2 six-chain monomer and several peaks indicative of aggregates. The table below the profile provides the results of an analysis of the several peaks. The FIT-Ig2 six-chain monomer constituted less than 75% of the purified protein. See, Example 1.7 for details.

FIG. 4 shows a size exclusion chromatography elution profile for a sample of FIT-Ig3 that had previously been purified by Protein A affinity chromatography. The elution profile is complex and indicates a significant proportion of aggregates. The FIT-Ig3 six-chain monomer constituted less than 40% of the purified protein. See, Example 1.7 for details.

FIG. 5 shows a size exclusion chromatography elution profile for a sample of FIT-Ig4 that had previously been purified by Protein A affinity chromatography. The elution profile shows a major peak of the FIT-Ig4 six-chain monomer and some peaks indicative of a low proportion of aggregates. The table below the profile provides the results of an analysis of the several peaks. The FIT-Ig4 six-chain monomer constituted approximately 91% of the purified protein. See, Example 1.7 for details.

FIG. 6 shows a size exclusion chromatography elution profile for a sample of FIT-Ig5 that had previously been purified by Protein A affinity chromatography. The elution profile shows a major peak of the FIT-Ig5 six-chain monomer and a minor peak indicative of a very low proportion of aggregates. The table below the profile provides the results of an analysis of the several peaks. The FIT-Ig5 six-chain monomer constituted greater than 98% of the purified protein. See, Example 1.7 for details.

FIG. 7 shows a size exclusion chromatography elution profile for a sample of FIT-Ig6 that had previously been purified by Protein A affinity chromatography. The elution profile shows a major peak of the FIT-Ig6 six-chain monomer and a barely detectable peak that may indicate an exceptionally low, if any, presence of aggregates. The table below the profile provides the results of an analysis of the several peaks. The FIT-Ig6 six-chain monomer constituted a surprising 99.9% of the purified protein. See, Example 1.7 for details.

FIG. 8 shows a graph of the serum concentration of FIT-Ig6 over time in three male Sprague-Dawley rats. FIT-Ig6 was administered to each rat as an intravenous dose of 5 mg/kg of body weight. See, Example 3 for details.

### DETAILED DESCRIPTION OF THE INVENTION

The Fabs-In-Tandem Immunoglobulin (“FIT-Ig”) binding protein format has been shown to be highly adaptable for providing bispecific, multivalent binding proteins directed to a wide variety of pairs of different target antigens or different epitopes on the same antigen. See, for example, International Publication Nos. WO 2015/103072 A1 and WO 2017/136820 A2. In a preferred format, a FIT-Ig binding protein comprises four Fab binding units (instead of two, as in a natural IgG antibody) in which each of two of the Fab units binds a first antigen (or epitope) and each of the other two Fab units binds a second antigen (or epitope). Despite success in using the FIT-Ig format to produce a diverse population of bispecific binding proteins that bind therapeutically relevant pairs of target antigens, a FIT-Ig binding protein that binds PD-L1 and EGFR has not previously been produced in quantity and quality that are suitable for conducting routine pre-clinical stage assessments as a candidate anti-cancer therapeutic drug. For example, as shown herein, a number of FIT-Ig binding proteins that bind PD-L1 and EGFR are not expressed in sufficiently high levels, i.e., greater than 10 mg/L, in standard mammalian cell cultures to provide sufficient amounts of the proteins required to support pre-clinical assessment including, for example, standard chemistry, manufacturing, and control (“CMC”) stage assessments. Another problem has been that previously produced FIT-Ig binding protein that bind PD-L1 and EGFR have exhibited a significant amount of aggregate formation, which substantially reduces the amount of functional, six-polypeptide chain, EGFR and PD-L1 binding protein “monomer” required for drug development. Unacceptable levels of FIT-Ig aggregates became evident in fractions of FIT-Ig binding protein eluted from Protein A affinity chromatography. Cost analyses revealed that none of the prior FIT-Ig binding protein constructs could be obtained in adequate quantity and quality to make a pre-clinical stage anti-cancer evaluation feasible.

This invention is based on the discovery of an EGFR/PD-L1 FIT-Ig binding protein that is expressed at sufficiently high levels in mammalian cell cultures and with no significant level of aggregate formation as to enable pre-clinical and clinical assessments as a therapeutic anti-cancer drug.

FIT-Ig binding proteins described herein comprise two or more antigen binding sites and are typically tetravalent (four antigen binding sites) proteins. A preferred FIT-Ig binding protein according to this invention binds both EGFR and PD-L1 and, therefore, is bispecific. Schematically, in a FIT-Ig binding protein, two first (heavy) polypeptide chains, each having the general structure “V-C-V-C-Fc”, where “V” is an antibody variable domain and C is an antibody constant domain, and four “light” polypeptide chains, each having the general structure “V-C”, associate to form a hexamer exhibiting four Fab binding units (VH-CH1 paired with VL-CL, sometimes notated VH-CH1::VL-CL). Each Fab binding unit comprises an antigen-binding site that comprises a heavy chain variable (VH) domain and a light chain variable (VL) domain with a total of six CDRs per antigen binding site. Thus, each half of a FIT-Ig binding protein comprises a heavy polypeptide chain and two light polypeptide chains, and complementary immunoglobulin pairing of the VH-CH1 and VL-CL elements of the three chains results in two Fab binding units, arranged in tandem. In the present invention, the immunoglobulin domains of the Fab binding units are directly fused in the heavy chain polypeptide, without the use of artificial interdomain linkers. That is, the N-terminal V-C element of each of the heavy polypeptide chains is directly fused at its C-terminus to the N-terminus of another V-C element, which in turn is linked to a C-terminal antibody Fc region. In bispecific FIT-Ig binding proteins, the tandem Fab binding units will be reactive with different antigens.

A description of the design, expression, and characterization of FIT-Ig binding proteins is provided in International Publication No. WO 2015/103072. A preferred example of such FIT-Ig molecules described herein comprises a heavy polypeptide chain and two different light polypeptide chains. The heavy chain comprises the structural formula VL_{A}-CL-VH_{B}-CH1-Fc where CL is directly fused to VH_{B} or VH_{B}-CH1-VL_{A}-CL-Fc where CH1 is directly fused to VL_{A}, wherein VL_{A} is a variable light domain from a parental antibody that binds antigen A, VH_{B} is a variable heavy domain from a parental antibody that binds antigen B, CL is a human IgG1 light chain kappa constant domain, CH1 is a first human IgG1 antibody heavy chain constant domain, and Fc is an immunoglobulin Fc region (e.g., the C-terminal hinge-CH2-CH3 portion of a heavy chain of a human IgG1 antibody). The two light polypeptide chains of the FIT-Ig have the formulas VH_{A}-CH1 and VL_{B}-CL, respectively. In bispecific FIT-Ig embodiments, antigen A and antigen B are different antigens, or different epitopes of the same antigen. In the present invention, one of A and B is human EGFR and the other is human PD-L1. Most preferably, in the present invention, A is human EGFR and B is human PD-L1.

Using the above scheme, each V domain of each polypeptide chain of a FIT-Ig binding protein may be designated with the antigen (or epitope) specificity of the antigen binding site from which it was derived. For example, using this “antigen-specificity” designation scheme, the structure of the first polypeptide chain (polypeptide chain #1) of the FIT-Ig6 binding protein described in Example 1.6 and Table 6, may be designated “VL_{EGFR}-CL-VH_{PD-L1}-CH1-Fc, wherein “VL_{EGFR}” indicates that the VL domain is derived from an EGFR-specific antigen binding site of an anti-EGFR parental antibody, and “VH_{PD-L1}” indicates that the VH domain is derived from a PD-L1-specific antigen binding site of an anti-PD-L1 parental antibody. The structure of the second polypeptide chain (polypeptide chain #2) of FIT-Ig6 may be designated “VH_{EGFR}-CH1”, wherein VH_{EGFR} indicates that the VH domain is derived from the EGFR-specific antigen binding site of the anti-EGFR parental antibody. Similarly, the third polypeptide chain (polypeptide chain #3) of FIT-Ig6 may be designated “VL_{PD-L1}-CL”, wherein “VL_{PD-L1}” indicates that the VL domain is derived from the PD-L1-specific antigen binding site of the anti-PD-L1 parental antibody. Thus, this antigen-specificity designation scheme indicates whether a particular antigen binding specificity is located in the corresponding outer or inner Fab binding units of the FIT-Ig binding protein.

In an alternative designation scheme, instead of designating the antigen specificity of individual variable domains of an antigen binding site (e.g., VL_{EGFR}, VH_{EGFR}, VL_{PD-L1}, VH_{PD-L1}), an abbreviated name of a parental antibody that served as the source of the domain is designated as a subscript of the respective VL and VH domains of an antigen binding site of a parental antibody. For example, referring again to the FIT-Ig6 binding protein of the invention described in Example 1.6 and Table 6, below, the structure of the first polypeptide chain (polypeptide chain #1) may be designated VLₚₐₙᵢ-CL-VH_{3G10}-CH1-Fc, wherein “VLₚₐₙᵢ” indicates that VL domain is derived from the anti-EGFR monoclonal antibody panitumumab and “VH_{3G10}” indicates that the VH domain is derived from the anti-PD-L1 monoclonal antibody 3G10. The structure of the second polypeptide chain (polypeptide chain #2) of FIT-Ig6 may be designated “VHₚₐₙᵢ-CH1”, wherein “VHₚₐₙᵢ” indicates that the VH domain is derived from panitumumab. Similarly, the third polypeptide chain (polypeptide chain #3) of FIT-Ig6 may be designated “VL_{3G10}-CL”, wherein “VL_{3G10}” indicates that the VL domain is derived from monoclonal antibody 3G10. Thus, this alternative designation scheme indicates the source of the antigen binding specificity of the outer and inner Fab binding units of the FIT-Ig binding protein. This source designation scheme is particularly useful when seeking to compare the properties of multiple FIT-Ig binding proteins that bind the same two antigens (or epitopes), but differ by one or both parental antibodies used as the source of antigen-binding specificities. See, the Examples below.

Association of a heavy polypeptide chain and each of the two different light polypeptide chains as described above forms two complete Fab binding units, each containing a typical antibody VH::VL antigen binding site. As with natural IgG antibodies, the Fc region on the heavy chain will associate with the Fc region on another heavy chain to form a homodimer and thereby provide a FIT-Ig binding protein that comprises six-polypeptide chains and four Fab binding units. Each arm of the FIT-Ig binding protein has an amino terminal or “outer” Fab binding unit and a carboxy proximal or “inner” Fab binding unit. In the FIT-Ig nomenclature adopted herein, a particular bispecific FIT-Ig binding protein may be designated with a prefix that indicates first the antigen specificity of the outer Fab binding units and then the antigen specificity of the inner Fab binding units. Thus, “EGFR/PD-L1 FIT-Ig binding protein” refers to a FIT-Ig binding protein that has two outer Fab binding units that bind EGFR and two inner Fab binding units that bind PD-L1.

Generally, nomenclatures used in connection with, and techniques of, cell and tissue culture, molecular biology, immunology, microbiology, oncology, genetics, and biochemistry are those that are well known and commonly used in the art. The methods and techniques of the present invention are generally performed according to conventional methods that are well known in the art and as described in various general and more specific references that are cited and discussed throughout the present specification unless otherwise indicated. Enzymatic reactions and purification techniques are performed according to manufacturer's specifications, as commonly accomplished in the art, or as described herein. The nomenclatures used in connection with, and the laboratory procedures and techniques of, analytical chemistry, synthetic organic chemistry, and medicinal and pharmaceutical chemistry described herein are those that are well known and commonly used in the art. Standard techniques are used for chemical syntheses, chemical analyses, pharmaceutical preparation, formulation, delivery, and treatment of patients.

So that the present invention may be more readily understood, select terms are defined below.

A “tumor” is an abnormal mass of tissue.

A “benign” tumor is an abnormal mass of tissue that grows slowly and is self-limiting in that it does not have the capacity to invade nearby tissues and spread beyond its original site. A benign tumor is not a cancer.

The term “cancer” has the meaning known in the fields of medicine and oncology, and includes the definition according to the National Cancer Institute (“NCI”, a division of the National Institutes of Health, Bethesda, Md., United States of America). Thus, according to the NCI, the term “cancer” is a term for diseases in which abnormal cells divide without control and that can invade and damage or destroy nearby tissues. Cancer cells can also slough off of primary tumors and spread (metastasize) to other parts of the body through the blood and/or lymph systems and establish “secondary tumors,” which are also referred to as “metastatic tumors” or “metastatic cancer”. Thus, the term “cancer” refers to a malignant tumor in which its cells grow uncontrollably and can penetrate and damage or destroy adjacent tissue, and that can metastasize through the circulation to distant parts of the body and form new tumors.

An “anti-cancer” compound or drug is one that blocks, inhibits, or halts the growth of cancer cells. A preferred anti-cancer compound is cytotoxic to cancer cells.

Unless otherwise differentiated, the terms “intravenous” (or “intravenously”) and “systemic” (or “systemically”) are used interchangeably with respect to a route for introducing a compound or a composition of the invention into the circulatory system of a cancer patient.

As used herein, the terms “treatment” and “treating” generally refer to any regimen that alleviates one or more symptoms or manifestations of cancer, that inhibits progression of cancer, that arrests progression or reverses progression of a cancer, that prevents onset of secondary (metastatic) cancer, that provides significant killing of metastatic cancer cells, that decreases the size of a primary or secondary (metastatic) cancer tumor, that increases remission of one or more secondary (metastatic) tumors over a period of time, that slows progression of primary or secondary (metastatic) tumors, that decreases the number of secondary (metastatic) tumors over a period of time, that decreases the number of new secondary (metastatic) tumors over a period of time, that increases organ or tissue function in a patient suffering from a cancer, that increases the vigor of a patient suffering from a cancer, that prolongs the life of a patient, or combinations thereof.

“Metastasis” has the same meaning known and used by persons skilled in the fields of oncology or medicine and refers to the process in which cancer cells spread from a primary tumor to another location in a patient's body. “Metastatic” cancer cells are cancer cells that have sloughed off or otherwise detached from a primary tumor and are in the process of traveling or already have traveled, usually through the blood or lymph, from the primary tumor to another location in a patient's body. In such a case, the cancer or its cells are said to have “metastasized”. A “metastatic” tumor is thus a tumor that has developed as the result of metastatic cancer cells traveling from a primary tumor to a different location in a patient's body where the cancer cells have established another (“secondary”, “metastatic”) tumor, which is the same type of tumor as the primary tumor. For example, a metastatic intestinal tumor in the liver is initiated by and composed of intestinal cancer cells that have metastasized from a primary intestinal tumor and traveled through the blood to the liver, where the cancer cells then established a secondary (metastatic) intestinal tumor. It is also understood that a metastatic tumor can also be a further source of metastatic cancer cells that can travel to other tissues and organs and establish additional metastatic tumors.

Unless indicated otherwise, when the terms “about” and “approximately” are used in combination with an amount, number, or value, then that combination describes the recited amount, number, integer, or value alone as well as the amount, number, or value plus or minus 5% of that amount, number, or value. By way of example, the phrases “about 40” and “approximately 40” disclose both “40” and “from 38 to 42, including 38 and 40”.

The term “polypeptide” refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein amino acid sequence. The term “polypeptide” encompasses fragments and variants (including fragments of variants) thereof, unless otherwise contradicted by context. For an antigenic polypeptide, a fragment of polypeptide optionally contains at least one contiguous or nonlinear epitope of polypeptide. The precise boundaries of the at least one epitope fragment can be confirmed using ordinary skill in the art. The fragment comprises at least about 5 contiguous amino acids, such as at least about 8 contiguous amino acids, at least about 10 contiguous amino acids, at least about 15 contiguous amino acids, or at least about 20 contiguous amino acids.

The term “isolated protein” or “isolated polypeptide” is a protein or polypeptide that by virtue of its origin or source of derivation is not associated with naturally associated components that accompany it in its native state, is substantially free of other proteins from the same species, is expressed by a cell from a different species, or does not occur in nature. Thus, a polypeptide that is chemically synthesized or synthesized in a cellular system different from the cell from which it naturally originates will be “isolated” from its naturally associated components. A protein consisting of one or more polypeptide chains may also be rendered substantially free of naturally associated components by isolation, using protein purification techniques well known in the art.

The term “recovering” refers to the process of rendering a chemical species such as a polypeptide substantially free of naturally associated components by isolation, e.g., using protein purification techniques well known in the art.

The term “biological activity” of PD-L1 or of EGFR refers to any or all inherent biological properties of PD-L1 or EGFR, respectively.

The term “specific binding” or “specifically binding” in reference to the interaction of an antibody, a binding protein, or a peptide with a second chemical species, means that the interaction is dependent upon the presence of a particular structure (e.g., an antigenic determinant or epitope) on the second chemical species. For example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. Bispecific FIT-Ig binding protein described herein comprise two Fab binding units that specifically bind EGFR and two Fab binding units that specifically bind PD-L1.

The term “antibody” broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below.

In a full-length antibody, each heavy chain is comprised of a heavy chain variable region (abbreviated herein as VH) and a heavy chain constant region. The heavy chain constant region is comprised of three domains: CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is comprised of three CDRs and four FRs, arranged from amino-terminus to carboxy-terminus in the following order: FR1, CDR1, FR2, CDR2, FR3, CDR3, FR4. First, second and third CDRs of a VH domain are commonly enumerated as CDR-H1, CDR-H2, and CDR-H3; likewise, first, second and third CDRs of a VL domain are commonly enumerated as CDR-L1, CDR-L2, and CDR-L3 Immunoglobulin molecules can be of any type (e.g., IgG, IgE, IgM, IgD, IgA and IgY), class (e.g., IgG1, IgG2, IgG3, IgG4, IgA1 and IgA2) or subclass.

In general, the term “Fc region”, or simply “Fc”, refers to the C-terminal region of an antibody heavy chain, which may be generated by papain digestion of an intact antibody. The Fc region may be a native sequence Fc region or a variant Fc region. The Fc region generally comprises two constant domains, i.e., a CH2 domain and a CH3 domain, and optionally comprises a CH4 domain, for example, as in the case of the Fc regions of IgM and IgE antibodies. The Fc region of IgG, IgA, and IgD antibodies comprises a hinge region, a CH2 domain, and a CH3 domain. In contrast, the Fc region of IgM and IgE antibodies lacks a hinge region but comprises a CH2 domain, a CH3 domain and a CH4 domain. Variant Fc regions having replacements of amino acid residues in the Fc portion to alter antibody effector function are known in the art (see, e.g., Winter et al., U.S. Pat. Nos. 5,648,260 and 5,624,821). Unless otherwise indicated, an “Fc region” of a FIT-Ig binding protein described herein is an Fc region that is derived from a human IgG1 antibody, that comprises a hinge region, CH2 domain, and CH3 domain, and that has an amino acid disclosed herein (SEQ ID NO:8) in any of Tables 1-6 in the Examples below.

The Fc region of an antibody mediates several important effector functions, for example, cytokine induction, antibody-dependent cell-mediated cytotoxicity (ADCC), phagocytosis, complement dependent cytotoxicity (CDC), and half-life/clearance rate of antibody and antigen-antibody complexes. In some cases, these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to Fc gamma receptors (FcγRs) and complement C1q, respectively. Unless indicated otherwise, the Fc region used in a FIT-Ig binding protein described herein retains at least one or more or all of the same functional properties as the Fc region had in its original donor antibody

In an embodiment, at least one amino acid residue is replaced in an Fc region such that one or more effector functions of the antibody are altered. As in an IgG antibody, the dimerization of two identical heavy chains of a FIT-Ig binding protein described herein is mediated by the dimerization of CH3 domains and is stabilized by the disulfide bonds within the hinge region that connects either a CH1 or CL of the FIT-Ig heavy chain to the Fc constant domains (e.g., CH2 and CH3). The anti-inflammatory activity of IgG is completely dependent on sialylation of the N-linked glycan of the IgG Fc fragment. The precise glycan requirements for anti-inflammatory activity have been determined, such that an appropriate IgG1 Fc fragment can be created, thereby generating a fully recombinant, sialylated IgG1 Fc with greatly enhanced potency (see, Anthony et al., Science, 320:373-376 (2008)). Such a sialylated Fc region may be used in a FIT-Ig binding protein described herein.

The terms “antigen-binding portion” and “antigen-binding fragment” or “functional fragment” of an antibody are used interchangeably and refer to one or more fragments of an antibody that retain the ability to specifically bind to an antigen, i.e., the same antigen (e.g., EGFR, PD-L1) as the full-length antibody from which the portion or fragment is derived. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment (Fab binding unit), a monovalent fragment consisting of the VL, VH, CL, and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature, 341: 544-546 (1989); International Publication No. WO 90/05144), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, for example, Bird et al., Science, 242: 423-426 (1988); and Huston et al., Proc. Natl. Acad. Sci. USA, 85: 5879-5883 (1988)). Such single chain antibodies are also intended to be encompassed within the term “antigen-binding portion” of an antibody and equivalent terms given above. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci. USA, 90: 6444-6448 (1993). Such antibody binding portions are known in the art (Kontermann and Dübel eds., Antibody Engineering (Springer-Verlag, New York, 2001), p. 790 (ISBN 3-540-41354-5)). In addition, single chain antibodies also include “linear antibodies” comprising a pair of tandem Fv segments (VH-CH1-VH-CH1) which, together with complementary light chain polypeptides, form a pair of antigen binding regions (Zapata et al., Protein Eng., 8(10): 1057-1062 (1995); and U.S. Pat. No. 5,641,870)).

Unless indicated otherwise, the terms “donor” and “parental” refer to any antibody or antigen-binding fragment that is a source for providing an antibody variable domain, an antibody constant domain, a Fab binding unit, or an Fc region for making a FIT-Ig binding protein described herein. A non-natural or engineered antibody may also serve as a donor or parent antibody for making a FIT-Ig binding protein described herein.

An antibody (or immunoglobulin) constant (C) domain refers to an antibody heavy (CH) or light (CL) chain constant domain. Murine and human immunoglobulin heavy chain and light chain constant domain amino acid sequences are known in the art.

The term “monoclonal antibody” or “mAb” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic determinant (epitope). Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “human antibody” includes antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created, or isolated by recombinant means, such as antibodies expressed using a recombinant expression vector transfected into a host cell, antibodies isolated from a recombinant, combinatorial human antibody library (Hoogenboom, H. R., Trends Biotechnol., 15: 62-70 (1997); Azzazy and Highsmith, Clin. Biochem., 35: 425-445 (2002); Gavilondo and Larrick, BioTechniques, 29: 128-145 (2000); Hoogenboom and Chames, Immunol. Today, 21: 371-378 (2000)), antibodies isolated from an animal (e.g., a mouse) that is transgenic for human immunoglobulin genes (see, e.g., Taylor et al., Nucl. Acids Res., 20: 6287-6295 (1992); Kellermann and Green, Curr. Opin. Biotechnol., 13: 593-597 (2002); Little et al., Immunol. Today, 21: 364-370 (2000)); or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “multivalent binding protein” denotes a binding protein comprising two or more antigen binding sites. A multivalent binding protein is preferably engineered to have three or more antigen binding sites, and is generally not a naturally occurring antibody. The term “bispecific binding protein” refers to a binding protein capable of binding two targets of different specificity.

The term “activity” includes properties such as the ability to bind a target antigen with specificity, the affinity of an antibody or binding protein for an antigen, the ability to neutralize the biological activity of a target antigen, the ability to inhibit interaction of a target antigen with its natural receptor(s) or natural ligand(s), and the like. Activities of an EGFR/PD-L1 FIT-Ig binding protein of the present invention may include, but are not limited to, inhibiting EGFR binding to its cognate ligand (EGF), inhibiting EGFR signaling, inhibiting PD-L1 binding to PD-1, inhibiting PD-1/PD-L1 signaling, upregulating T cell response to cancer, killing cancer cells, inhibiting cancer cell growth, inhibiting cancer cell survival, and inhibiting spread of cancer cells.

The term “kon” (also “Kon”, “kon”), as used herein, is intended to refer to the on-rate constant for association of a binding protein (e.g., an antibody) to an antigen to form an association complex, e.g., antibody/antigen complex, as is known in the art. The “kon” also is known by the terms “association rate constant”, or “ka”, as used interchangeably herein. For example, this value can indicate the binding rate of an antibody to its target antigen or the rate of complex formation between an antibody and antigen as is shown by the equation below:

Antibody (“Ab”)+Antigen (“Ag”)→Ab-Ag.

The term “koff” (also “Koff”, “koff”), as used herein, is intended to refer to the off-rate constant for dissociation, or “dissociation rate constant”, of a binding protein (e.g., an antibody) from an association complex (e.g., an antibody/antigen complex) as is known in the art. For example, this value can indicate the dissociation rate of an antibody from its target antigen or separation of Ab-Ag complex over time into free antibody and antigen as shown by the equation below:

Ab+Ag←Ab-Ag.

The term “K_{D}” (also “Kd”), as used herein, is intended to refer to the “equilibrium dissociation constant”, and refers to the value obtained in a titration measurement at equilibrium, or by dividing the dissociation rate constant (k_{off}) by the association rate constant (kₒₙ). The association rate constant (kₒₙ), the dissociation rate constant (k_{off}), and the equilibrium dissociation constant (K_{D}) are used to represent the binding affinity of an antibody or binding protein to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Biolayer interferometry (BLI) using, e.g., the Octet® RED96 system (Pall FortéBio LLC), is another affinity assay technique. Additionally, a KinExA® (Kinetic Exclusion Assay) assay (available from Sapidyne Instruments, Boise, Id.) can also be used.

The term “isolated nucleic acid” shall mean a polynucleotide (e.g., of genomic, cDNA, or synthetic origin, or some combination thereof) that, by human intervention, is not associated with all or a portion of the polynucleotides with which it is found in nature, is operably linked to a polynucleotide that it is not linked to in nature, or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

The term “recombinant host cell” (or simply “host cell”), is intended to refer to a cell into which exogenous DNA has been introduced. In an embodiment, the host cell comprises two or more (e.g., multiple) nucleic acids encoding antibodies, such as the host cells described in U.S. Pat. No. 7,262,028, for example. Such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the Kingdoms of life. In another embodiment, eukaryotic cells include protist, fungal, plant and animal cells. In another embodiment, host cells include but are not limited to the prokaryotic cell line Escherichia coli; mammalian cell lines CHO, HEK293, COS, NS0, SP2 and PER.C6; the insect cell line Sf9; and the fungal cell Saccharomyces cerevisiae.

Standard techniques may be used for recombinant DNA, oligonucleotide synthesis, cell culture, tissue culture, and transformation (e.g., transfection, electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., Molecular Cloning: A Laboratory Manual, 2nd ed. (Cold Spring Harbor Laboratory Press, Cold Spring Harbor, N.Y., 1989).

The term “agonist”, as used herein, refers to a modulator that, when contacted with a molecule of interest, causes an increase in the magnitude of a certain activity or function of the molecule compared to the magnitude of the activity or function observed in the absence of the agonist. The terms “antagonist” and “inhibitor”, as used herein, refer to a modulator that, when contacted with a molecule of interest causes a decrease in the magnitude of a certain activity or function of the molecule compared to the magnitude of the activity or function observed in the absence of the antagonist. A particular antagonist of the invention is an EGFR/PD-L1 FIT-Ig described herein that blocks or inhibits EGFR binding to EGF, that blocks or inhibits PD-L1 binding to PD-1, that blocks or inhibits EGFR signaling, that blocks or inhibits PD-L1 signaling, that blocks or inhibits the cancer-promoting activities of EGFR-dependent signaling, that blocks or inhibits cancer-promoting activities of PD-L1-dependent signaling, and one or more combinations thereof.

As used herein, the term “effective amount” refers to the amount of a therapy that is sufficient to reduce or ameliorate the severity and/or duration of a disorder or one or more symptoms thereof; prevent the advancement of a disorder; cause regression of a disorder; prevent the recurrence, development, or progression of one or more symptoms associated with a disorder; detect a disorder; or enhance or improve the prophylactic or therapeutic effect(s) of another therapy (e.g., prophylactic or therapeutic agent).

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

A composition or method described herein as “comprising” one or more named elements or steps is open-ended, meaning that the named elements or steps are essential, but other elements or steps may be added within the scope of the composition or method. To avoid prolixity, it is also understood that any composition or method described herein as “comprising” (or “which comprises”) one or more named elements or steps also describes the corresponding, more limited, composition or method “consisting essentially of” (or “which consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and close-ended composition or method “consisting of” (or “which consists of”) the named elements or steps to the exclusion of any other unnamed element or step. In any composition or method disclosed herein, known or disclosed equivalents of any named essential element or step may be substituted for that element or step.

### Features of a Preferred EGFR/PD-L1 FIT-Ig Binding Protein of the Invention

A preferred EGFR/PD-L1 FIT-Ig binding protein of the invention referred to as “FIT-Ig6” (or “EGFR/PD-L1 FIT-Ig6”) binds EGFR and PD-L1 and comprises:

a first polypeptide chain (heavy chain) comprising an amino acid sequence according to SEQ ID NO:1;

a second polypeptide chain (first light chain) comprising an amino acid sequence according to SEQ ID NO:2;

a third polypeptide chain (second light chain) comprising an amino acid sequence according to SEQ ID NO:3.

The association of a first, second, and third polypeptide chain described above provides a “half-FIT” molecule comprising an amino terminal or “outer” Fab binding unit, which is specific for EGFR, linked in tandem to a carboxy proximal or “inner” Fab binding, which is specific for PD-L1. As in a natural IgG1 antibody, the Fc (hinge-CH2-CH3) at the carboxy terminal region of the first polypeptide chain can associate with the Fc of another half-FIT molecule to form the fully assembled, six-polypeptide chain FIT-Ig binding protein that comprises two outer EGFR-specific Fab binding units and two inner PD-L1-specific Fab binding units.

The specificity of a Fab binding unit of a FIT-Ig binding protein is derived from a parental antibody that is used as the source of antibody heavy and light chain variable domains (VH, VL) that form a specific antigen binding site.

An EGFR/PD-L1 FIT-Ig binding protein of the invention binds EGFR and PD-L1 simultaneously. In a further embodiment, the EGFR/PD-L1 FIT-Ig binding protein binds two EGFR proteins and two PD-L1 proteins simultaneously.

An EGFR/PD-L1 FIT-Ig binding protein according to the invention binds EGFR and PD-L1 with affinities similar to those of each of the individual parental antibodies from which the EGFR and PD-L1 specificities are derived.

The affinity of an EGFR/PD-L1 FIT-Ig binding protein according to the invention for EGFR and PD-L1 can be measured using any of a variety of systems, including biolayer interferometry (for example, using the Octet® RED96 system, Pall FortéBio LLC), surface plasmon resonance (for example, using a BIAcore® (biomolecular interaction analysis) assay system, BIAcore International AB, a GE Healthcare company, Uppsala, Sweden), or a kinetic exclusion assay (for example, using a KinExA® assay system, Sapidyne Instruments, Boise, Id.).

An EGFR/PD-L1 FIT-Ig binding protein of the invention binds EGFR and has an on-rate constant (kₒₙ) for human EGFR that is at least 3×10⁵ M⁻¹s⁻¹, more preferably at least 2×10⁵ M⁻¹s⁻¹, as determined by biolayer interferometry. In a further embodiment, an EGFR/PD-L1 FIT-Ig binding protein according to this invention has a kₒₙ for human EGFR that is approximately 40% lower than the kₒₙ for human EGFR of the parental antibody from which the anti-EGFR specificity of the EGFR/PD-L1 FTI-Ig binding protein was derived.

An EGFR/PD-L1 FIT-Ig binding protein of the invention binds human EGFR and has an off-rate constant (k_{off}) for human EGFR that is less than 1.1×10⁻⁴ sec⁻¹, as determined by biolayer interferometry. In a further embodiment, an EGFR/PD-L1 FIT-Ig binding protein of the invention has a k_{off} for human EGFR that is approximately 50% lower than the value of the k_{off} for human EGFR of the parental antibody from which the anti-EGFR specificity of the EGFR/PD-L1 FIT-Ig binding protein was derived.

An EGFR/PD-L1 FIT-Ig binding protein of the invention binds human EGFR and has a dissociation constant (K_{D}) for human EGFR that is less than 1×10⁻⁹ M, preferably less than 7×10⁻¹⁰ M, more preferably less than 6×10⁻¹⁰ M, and still more preferably less than or equal to 5×10⁻¹⁰ M as determined by biolayer interferometry. In a further embodiment, an EGFR/PD-L1 FIT-Ig binding protein of the invention has a K_{D} for human EGFR that is substantially the same as (i.e., identical to or within 25% of) the K_{D} for human EGFR of the parental antibody from which the anti-EGFR specificity of the EGFR/PD-L1 FIT-Ig binding protein was derived.

An EGFR/PD-L1 FIT-Ig binding protein of the invention binds PD-L1 and has an on-rate constant (kₒₙ) for human PD-L1 that is at least 5×10⁵ M⁻¹s⁻¹, more preferably at least 7×10⁵ M⁻¹s⁻¹, and still more preferably at least 8×10⁵ M⁻¹s⁻¹, as determined by biolayer interferometry. In a further embodiment, an EGFR/PD-L1 FIT-Ig binding protein according to this invention has a kₒₙ for human PD-L1 that is the same or within approximately 90% of the kₒₙ for human PD-L1 of the parental antibody from which the anti-PD-L1 specificity of the EGFR/PD-L1 FTI-Ig binding protein was derived.

An EGFR/PD-L1 FIT-Ig binding protein of the invention binds human PD-L1 and has an off-rate constant (k_{off}) for human PD-L1 that is less than 2×10⁻² sec⁻¹ and more preferably less than 1.5×10⁻² sec⁻¹ as determined by biolayer interferometry. In a further embodiment, an EGFR/PD-L1 FIT-Ig binding protein of the invention has a k_{off} for human PD-L1 that is approximately 20% higher than k_{off} for human PD-L1 of the parental antibody from which the anti-PD-L1 specificity of the EGFR/PD-L1 FIT-Ig binding protein was derived.

An embodiment, an EGFR/PD-L1 FIT-Ig binding protein of the invention binds human PD-L1 and has a dissociation constant (K_{D}) for PD-L1 that is less than 2×10⁻⁸ M and more preferably less than 1.7×10⁻⁸ M as determined by biolayer interferometry. In a further embodiment, an EGFR/PD-L1 FIT-Ig binding protein of the invention has a K_{D} for PD-L1 that is substantially the same as (i.e., identical to or within 30% of) the K_{D} for PD-L1 of the parental antibody from which the anti-PD-L1 specificity of the EGFR/PD-L1 FIT-Ig binding protein was derived.

A preferred EGFR/PD-L1 FIT-Ig binding protein of the invention exhibits no significant aggregate formation after a one-step purification from cell culture media using a Protein A affinity chromatography. As shown herein, after purification over a Protein A affinity chromatography, the column eluate containing EGFR/PD-L1 FIT-Ig binding protein can be analyzed for the presence of aggregates using size exclusion chromatography (e.g., size exclusion chromatography using a high performance liquid chromatography (HPLC) column). Size exclusion chromatography (SEC) will separate molecules based on size and therefore will separate molecules having the molecular weight expected for the fully assembled, six-polypeptide chain EGFR/PD-L1 FIT-Ig binding protein, also referred to as the six-chain “monomer”, from other species having higher or lower molecule weights. The six-chain monomer has a molecular weight of approximately 240,000 daltons. A preferred EGFR/PD-L1 FIT-Ig binding protein of the invention that has been purified from culture media using Protein A affinity chromatography has less than or equal to 0.1% aggregates. That is, 99.9% of the EGFR/PD-L1 FIT-Ig binding protein of the invention produced in mammalian cell culture will be present as a fully-assembled, six-chain monomer. A level of less than or equal to 0.1% (≤0.1%) of protein aggregates is considered an insignificant amount that does not prevent efficient pre-clinical and clinical assessments of the EGFR/PD-L1 FIT-Ig binding protein as an anti-cancer drug. In contrast, as shown herein, the amount of aggregates found in other previously produced EGFR/PD-L1 and PD-1/EGFR FIT-Ig binding proteins have ranged from 1.2% up to greater than 70%. Thus, with respect to aggregate formation, an EGFR/PD-L1 FIT-Ig binding protein of the invention is more stable and has a significantly lower (for example, at least a 10-fold lower) percentage of aggregates than previously produced FIT-Ig binding proteins that bind EGFR and PD-L1. See, Table 7 in Example 1.7, below.

### Production of an EGFR/PD-L1 FIT-Ig Binding Protein of the Invention

The invention provides methods of producing an EGFR/PD-L1 FIT-Ig binding protein described herein comprising culturing an isolated host cell comprising one or more vectors encoding the three polypeptide chains of the EGFR/PD-L1 FIT-Ig binding protein under conditions sufficient to produce an EGFR/PD-L1 FIT-Ig binding protein. The desired EGFR/PD-L1 FIT-Ig binding protein is expressed as a six-polypeptide chain FIT-Ig binding protein that comprises two outer EGFR-specific Fab binding units and two inner PD-L1-specific Fab binding units.

A variety of expression systems comprising expression vectors and compatible prokaryotic or eukaryotic host cells are available for expressing recombinant heterologous proteins. An example of a prokaryotic host cell that is often used to express a recombinant protein is an Escherichia coli cell. Eukaryotic host cells that may be used to express recombinant proteins include, without limitation, mammalian host cells, insect host cells, plant host cells, fungal host cells, algal host cells, nematode host cells, protozoan host cells, and fish host cells. Fungal host cells that may be used for expressing recombinant proteins include, but are not limited to: Aspergillus, Neurospora, Saccharomyces, Pichia, Hansenula, Schizosaccharomyces, Kluyveromyces, Yarrowia, and Candida. A preferred Saccharomyces host cell for expressing recombinant proteins is a Saccharomyces cerevisiae cell. An insect cell useful as a host cell according to the invention is an insect Sf9 cell.

FIT-Ig binding proteins are preferably produced using mammalian cell expression systems. The construction and expression of FIT-Ig binding proteins has been previously described in International Publication Nos. WO 2015/103072 A1 and WO 2017/136820 A2. An EGFR/PD-L1 FIT-Ig binding protein of the invention can be produced using similar materials and methods. Such methods are commonly employed for expressing recombinant antibodies and engineered binding proteins in selected host cells.

Typically, each polypeptide chain of a FIT-Ig binding protein is encoded on an isolated nucleic acid molecule along with an amino terminal signal sequence (signal peptide), which directs the nascent polypeptide chain into the lumen of the endoplasmic reticulum (ER) and then into the Golgi apparatus for secretion. Individual nucleic acid molecules encoding polypeptide chains of a FIT-Ig binding protein described herein can be made using chemical DNA synthesis methods, using recombinant DNA methods, or using a combination of both methodologies.

Each nucleic acid molecule encoding one of the polypeptide chains of a FIT-Ig binding protein is then inserted into a separate expression vector and thereby operably linked to appropriate transcriptional and/or translational sequences that permit expression of the polypeptide chain in a host cell that is compatible with the expression vector.

An expression vector may be an autonomously replicating vector or a vector that incorporates the isolated nucleic acid that is present in the vector into a host cell genome. Preferred vectors for expressing nucleic acids described herein include, but are not limited to, pcDNA, pTT (Durocher et al, Nucleic Acids Res., 30(2e9): 1-9 (2002)), pTT3 (pTT with additional multiple cloning sites), pEFBOS (Mizushima and Nagata, Nucleic Acids Res., 18(17): 5322 (1990)), pBV, pJV, pcDNA3.1 TOPO, pEF6 TOPO, pBJ, and modifications thereof as required for expressing an EGFR/PD-L1 FIT-Ig binding protein described herein in a particular host cell.

In the preferred protocol for producing FIT-Ig binding proteins, a FIT-Ig binding protein is expressed in a mammalian host cell transfected with three expression vectors, wherein each expression vector comprises a nucleic acid encoding one of the three component polypeptide chains of the FIT-Ig binding protein.

Preferably, an isolated mammalian host cell comprising a vector described herein is selected from the group consisting of: a Chinese Hamster Ovary (CHO) cell, a COS cell, a Vero cell, an SP2/0 cell, an NS/0 myeloma cell, a human embryonic kidney (HEK293) cell, a baby hamster kidney (BHK) cell, a HeLa cell, a human B cell, a CV-1/EBNA cell, an L cell, a 3T3 cell, an HEPG2 cell, a PerC6 cell, and an MDCK cell.

Transfected HEK293 cells are routinely used as a transient transfection system, which can provide short term production of recombinant proteins, including engineered antibodies and binding proteins, for example, from 4 to 10 days post-transfection. Transfected HEK293 cells are routinely employed for initial laboratory cloning, production, and analysis of recombinant proteins, thereby avoiding the time and labor required to isolate a stably transfected production cell line, such as a stable transfected CHO cell line. To practitioners familiar with production of engineered antibodies and binding proteins, a level of expression of less than 10 mg/L in cultures of transiently transfected cells is too low to expect that the adequate amounts of the binding protein will be available to conduct initial pre-clinical assessments, for example, biological activity studies, preliminary stability studies, pharmacokinetic (PK) studies, and efficacy in animal models. In addition, practitioners in this field also recognize that a level of expression of less than 10 mg/L in cultures of transiently transfected HEK293 cells indicates that the isolation of a stably transfected CHO cell with high expression (for example, greater than 1 g/L) is not likely to be successful, even with extensive investments of time and labor. In contrast, an expression level for a FIT-Ig binding protein of greater than 10 mg/L in cultures of transiently transfected HEK293 cells is considered sufficiently high to provide amounts of the binding protein to conduct early discovery stage assessments prior to generation of a stably transfected CHO cell and also indicative of a likely successful isolation of a stably transfected CHO cell required for producing much higher amounts required for both later pre-clinical stage and clinical stage assessments.

As shown herein, an EGFR/PD-L1 FIT-Ig binding protein according to the invention can be expressed in cultures of transfected HEK293 cells at levels greater than 10 mg of EGFR/PD-L1 FIT-Ig binding protein per liter of cell culture (>10 mg/L). This level of expression was unexpected in view of the fact that previously produced EGFR/PD-L1 and PD-1/EGFR FIT-Ig binding proteins were only expressed at levels ranging from about 1 mg/L to about 8 mg/L. In addition, more than an incremental enhancement in the level of production compared to that of other FIT-Ig binding proteins, the level of expression of an EGFR/PD-L1 FIT-Ig binding protein of the invention in cultures of transfected HEK293 cells ensures that the binding protein is available in sufficient amounts to undergo both pre-clinical stage and clinical stage assessments as a new anti-cancer therapeutic drug.

### Pharmaceutical Compositions

A pharmaceutical composition of the invention comprises an EGFR/PD-L1 FIT-Ig binding protein described herein and one or more pharmaceutically acceptable components such as a pharmaceutically acceptable carrier (vehicle, buffer), excipient, and/or other ingredient. By “pharmaceutically acceptable” is meant that a carrier, compound, component, or other ingredient of a composition is compatible with the physiology of a human subject and also is not deleterious to the desired binding specificity of the EGFR/PD-L1 FIT-Ig binding protein, or to any other desired property or activity of any other component that may be present in a composition that is to be administered to a human subject. Examples of pharmaceutically acceptable carriers that may be used in a pharmaceutical composition of the invention include, but are not limited to, water, saline, phosphate buffered saline, dextrose, glycerol, ethanol, and the like, as well as combinations thereof. In some cases, it may be preferable to include isotonic agents, including, but not limited to, sugars; polyalcohols, such as mannitol or sorbitol; sodium chloride; and combinations thereof.

Pharmaceutically acceptable compositions of the invention may further comprise one or more excipients, minor amounts of auxiliary substances such as wetting or emulsifying agents, fillers, preservatives, or buffers to enhance the shelf life or effectiveness of the pharmaceutical composition. An excipient is generally any compound or combination of compounds that provides a beneficial property or feature to a pharmaceutical composition other than a primary therapeutic compound or activity. With respect to a pharmaceutical composition comprising an EGFR/PD-L1 FIT-Ig binding protein of the invention (primary therapeutic compound), an excipient provides a desired beneficial feature other than the desired binding specificity of the EGFR/PD-L1 FIT-Ig binding protein or anti-cancer activity owing to the EGFR/PD-L1 FTI-Ig binding protein.

In another embodiment, a pharmaceutical composition of the invention comprises an EGFR/PD-L1 FIT-Ig binding protein as described herein, a pharmaceutically acceptable carrier, and an adjuvant, wherein the adjuvant provides a general stimulation of the human immune system.

The pH may be adjusted in a pharmaceutical composition as necessary, for example, to promote or maintain solubility of component ingredients, to maintain stability of one or more component ingredients in the formulation, and/or to deter undesired growth of microorganisms that potentially may be introduced into the composition.

A pharmaceutical composition comprising an EGFR/PD-L1 FTI-Ig binding protein of the invention may be prepared to provide a sustained or time-delayed release of the binding protein. A variety of methods for the preparation of such controlled release or time-delayed compositions are known to those skilled in the art, including, but not limited to, implants, transdermal patches, and microencapsulated delivery systems. Biodegradable, biocompatible polymers can also be used, such as ethylene vinyl acetate, a polyanhydride, a polyglycolic acid, a collagen, a polyorthoester, a polylactic acid, and combinations thereof, to prepare controlled released or time-delayed compositions comprising an EGFR/PD-L1 FIT-Ig binding protein of the invention.

A pharmaceutical composition comprising an EGFR/PD-L1 FTI-Ig binding protein described herein may further comprise one or more additional therapeutically active compounds (therapeutic agents). Examples of such additional therapeutic agents that may be incorporated into a pharmaceutical composition of the invention include, but are not limited to, an anti-cancer agent that is different from an EGFR/PD-L1 FTI-Ig binding protein described herein (for example, a cytotoxic metal-containing anti-cancer compound or a cytotoxic radioisotope-based anti-cancer compound, and combinations thereof), an antibiotic, an anti-viral compound, a sedative, a stimulant, a local anesthetic, an anti-inflammatory steroid (for example, natural or synthetic anti-inflammatory steroids and combinations thereof), an analgesic (for example, acetylsalicylic acid, acetaminophen, naproxen, ibuprofen, a COX-2 inhibitor, morphine, oxycodone, and combinations thereof), an anti-histamine, a non-steroidal anti-inflammatory drug (“NSAID,” for example, acetylsalicylic acid, ibuprofen, naproxen, a COX-2 inhibitor, and combinations thereof), and combinations thereof.

A pharmaceutical composition according to the invention is formulated for administration by any of a variety of routes known in the art. Such routes, include but are not limited to, parenteral, intravenous (systemic), subcutaneous, intramuscular, oral (i.e., gastrointestinal), sub-lingual, buccal, intranasal (e.g., inhalation), transdermal (e.g., topical), intratumoral, transmucosal, intraarticular, intrabronchial, intracapsular, intracartilaginous, intracavitary, intracervical, intrahepatic, intramyocardial, intraosteal, intrapelvic, intrapericardial, intraperitoneal, intrapleural, intraprostatic, intrapulmonary, intrarenal, intraretinal, intraspinal, intrasynovial, intrathoracic, intrauterine, intravesical, vaginal, and rectal.

Preferably, a pharmaceutical composition according to the invention is formulated for intravenous administration to a human subject that has cancer. Intravenous administration of a pharmaceutical composition comprising an EGFR/PD-L1 FTI-Ig binding protein provides the EGFR/PD-L1 FTI-Ig binding protein throughout the circulatory system and thereby to tissues and organs reached by the circulating blood. Typically, compositions for intravenous administration are solutions in sterile isotonic aqueous buffer. Where necessary, the composition may also include a solubilizing agent and a local anesthetic, such as lignocamne, to ease pain at the site of the injection.

Subcutaneous administration of a pharmaceutical composition of the invention is a route by which the EGFR/PD-L1 FTI-Ig binding protein may be provided to the lymph system. Accordingly, a pharmaceutical composition comprising an EGFR/PD-L1 FTI-Ig binding protein of the invention may be formulated for subcutaneous administration.

A pharmaceutical composition of the invention may be formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions or solutions or emulsions in oily or aqueous vehicles, and may contain formulary agents such as suspending, stabilizing, and/or dispersing agents. Alternatively, the active ingredient (i.e., EGFR/PD-L1 FIT-Ig binding protein of the invention) may be in powder form (e.g., lyophilized form) for reconstitution with a suitable vehicle (e.g., sterile pyrogen-free water) before use.

A pharmaceutical composition of the invention may be formulated for delivery as a depot preparation as a type of long acting formulation. Such long acting formulations may be administered by implantation (e.g., subcutaneously or intramuscularly) or by intramuscular injection. Thus, for example, a pharmaceutical composition may be formulated with suitable polymeric or hydrophobic materials (e.g., as an emulsion in an acceptable oil) or ion exchange resins, or as sparingly soluble derivatives (e.g., as a sparingly soluble salt).

In another embodiment, an EGFR/PD-L1 FIT-Ig binding protein described herein may be a crystallized EGFR/PD-L1 FIT-Ig binding protein that retains binding activity for EGFR and PD-L1 bound by the non-crystallized EGFR/PD-L1 FIT-Ig binding protein. Such a crystallized EGFR/PD-L1 FIT-Ig binding protein may also provide carrier-free controlled release of the EGFR/PD-L1 FIT-Ig binding protein when administered to an individual. A crystallized EGFR/PD-L1 FIT-Ig binding protein of the invention may also exhibit a greater in vivo half-life when administered to an individual compared to the non-crystallized form. Crystallized binding proteins of the invention may be produced according to methods known in the art and as disclosed in International Publication No. WO 02/072636 (Shenoy et al.), incorporated herein by reference.

A pharmaceutical composition for the release of a crystallized EGFR/PD-L1 FIT-Ig binding protein wherein the composition comprises a crystallized EGFR/PD-L1 FIT-Ig binding protein as described herein, an excipient ingredient, and at least one polymeric carrier. Preferably the excipient ingredient is selected from the group consisting of: albumin, sucrose, trehalose, lactitol, gelatin, hydroxypropyl-β-cyclodextrin, methoxypolyethylene glycol and polyethylene glycol. Preferably the polymeric carrier is a polymer selected from one or more of the group consisting of: poly(acrylic acid), poly(cyanoacrylates), poly(amino acids), poly(anhydrides), poly(depsipeptide), poly(esters), poly(lactic acid), poly(lactic-co-glycolic acid) or PLGA, poly(b-hydroxybutryate), poly(caprolactone), poly(dioxanone); poly(ethylene glycol), poly((hydroxypropyl) methacrylamide, poly[(organo) phosphazene], poly(ortho esters), poly(vinyl alcohol), poly(vinylpyrrolidone), maleic anhydride/alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends thereof, and copolymers thereof.

### Methods and Uses of an EGFR/PD-L1 FIT-Ig Binding Protein of the Invention

The ability of the EGFR/PD-1 FIT-Ig binding protein to bind both EGFR and PD-L1 is primarily of interest in providing an anti-cancer therapy. Presumably, the binding of the EGFR/PD-L1 FIT-Ig binding protein to EGFR and PD-L1 inhibits or blocks EGFR and PD-L1 from binding to their respective ligands (e.g., EGFR and PD-1), which in turn inhibits or blocks the respective separate signaling pathways (i.e., EGFR/EGF signaling and PD-L1/PD-1 signaling), which are involved in carcinogenesis, cancer cell growth, and the spread of cancer cells (metastasis). An EGFR/PD-L1 FIT-Ig binding protein of the invention preferably is capable of blocking human EGFR or human PD-L1 signaling activity both in vitro and in vivo. Accordingly, the EGFR/PD-L1 FIT-Ig binding protein of the invention can be used to inhibit or block human EGFR and/or human PD-L1 signaling in human subjects or presumably in other mammalian subjects having an EGFR and a PD-L1 with which an EGFR/PD-L1 FIT-Ig binding protein of the invention cross-reacts.

A method of inhibiting or blocking EGFR signaling in a cell comprises contacting a cell expressing EGFR with an EGFR/PD-L1 FIT-Ig binding protein of the invention.

A method of inhibiting or blocking PD-L1 signaling in a cell comprises contacting a cell expressing PD-L1 with an EGFR/PD-L1 FIT-Ig binding.

A method of inhibiting or blocking EGFR signaling and PD-L1 signaling comprises a contacting a population of cells comprising cells expressing EGFR and cells expressing PD-L1 with an EGFR/PD-L1 FIT-Ig binding protein of the invention.

A FIT-Ig binding protein useful in the methods described above is an EGFR/PD-L1 FIT-Ig binding protein that comprises a first (heavy) polypeptide chain comprising an amino acid sequence according to SEQ ID NO:1; a second (first light) polypeptide chain comprising an amino acid sequence according to SEQ ID NO:2; and a third (second light) polypeptide chains comprising an amino acid sequence according to SEQ ID NO:3.

The invention also provides a method of treating a cancer in a human subject in need of treatment thereof, comprising administering to the subject an EGFR/PD-L1 FIT-Ig binding protein, or a pharmaceutical composition comprising the EGFR/PD-L1 FIT-Ig binding protein, wherein the EGFR/PD-L1 FIT-Ig binding protein comprises a first (heavy) polypeptide chain comprising an amino acid sequence according to SEQ ID NO:1; a second (first light) polypeptide chain comprising an amino acid sequence according to SEQ ID NO:2; and a third (second light) polypeptide chains comprising an amino acid sequence according to SEQ ID NO:3.

In a method of treating cancer in a human subject according to the invention, the cancer may be an epithelial cancer.

In another embodiment, the cancer that is treated in a method of the invention is selected from the group consisting of: a melanoma (e.g., metastatic malignant melanoma), renal cancer (e.g. clear cell carcinoma), prostate cancer (e.g. hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

In an embodiment, the invention also provides a method for restoring the activity of activated T cells (reversing suppression) comprising contacting human PD-L1-expressing cells with an EGFR/PD-L1 FIT-Ig binding protein of the invention such that PD-L1/PD-1 initiated T cell suppression is inhibited. In another embodiment, the invention provides a method for inhibiting carcinogenesis induced by EGFR/EGF binding, comprising contacting human EGFR-expressing cells with an EGFR/PD-L1 FIT-Ig binding protein of the invention such that EGFR/EGF-mediated signaling is inhibited or blocked.

In another embodiment, the invention provides a method for treating a human subject suffering from a disease in which EGFR and/or PD-L1 activity is detrimental, such method comprising administering to the subject an EGFR/PD-L1 binding protein of the invention such that an activity mediated by PD-L1/PD1 binding and/or EGFR/EGF binding in the subject is reduced.

A FIT-Ig binding protein useful in the methods described above is an EGFR/PD-L1 FIT-Ig binding protein that comprises a first (heavy) polypeptide chain comprising an amino acid sequence according to SEQ ID NO:1; a second (first light) polypeptide chain comprising an amino acid sequence according to SEQ ID NO:2; and a third (second light) polypeptide chains comprising an amino acid sequence according to SEQ ID NO:3.

As used herein, the term “a disease in which EGFR and/or PD-L1 activity is detrimental” is intended to include diseases in which the interaction of EGFR with its ligand (EGR) or the interaction of PD-L1 with its ligand (PD-1) in a subject suffering from the disorder is either responsible for the pathophysiology of the disorder or is a factor that contributes to a worsening of the disease. Accordingly, a disease in which EGFR and/or PD-L1 activity is detrimental is a disease in which inhibition of EGFR and/or PD-L1 activity is expected to alleviate the symptoms and/or progression of the disease.

Given that an EGFR/PD-L1-FIT-Ig binding protein of the invention binds to human EGFR and PD-L1, the EGFR/PD-L1 binding protein may also be used to detect EGFR or PD-L1, or both, e.g., in a biological sample containing cells that express one or both of those target antigens. For example, the EGFR/PD-L1 binding protein of the invention can be used in a conventional immunoassay, such as an enzyme linked immunosorbent assays (ELISA), a radioimmunoassay (RIA), or tissue immunohistochemistry. The invention provides a method for detecting EGFR or PD-L1 in a biological sample comprising contacting a biological sample with the EGFR/PD-L1 FIT-Ig binding protein of the invention and detecting whether binding to a target antigen (EGFR or PD-L1) occurs, thereby detecting the presence or absence of the target in the biological sample. The binding protein may be directly or indirectly labeled with a detectable substance to facilitate detection of the bound or unbound antibody/fragment/binding protein. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

Having now described the present invention in detail, the same will be more clearly understood by reference to the following examples, which are included for purposes of illustration only and are not intended to be limiting of the invention.

### EXAMPLES

### Example 1. Production of FIT-Ig Binding Proteins that Bind EGFR and PD-L1

Six bispecific Fabs-in-Tandem Immunoglobulin (FIT-Ig) binding proteins recognizing both human EGFR and human PD-L1 were constructed using binding sites from anti-PD-L1 and anti-EGFR parental antibodies.

Anti-PD-L1 monoclonal antibodies (mAbs) 1B12, 10A5, and 3G10 were previously described. See, for example, U.S. Pat. No. 7,943,743 B2.

The use of particular amino acid sequences of the anti-EGFR mAb panitumumab to make FIT-Ig binding proteins has been previously described. See, for example, International Publication No. WO 2017/136820 A2.

### Example 1.1: FIT-Ig1

A PD-L1/EGFR FIT-Ig designated “FIT-Ig1” (also referred to as “PD-L1/EGFR FIT-Ig1”) was constructed utilizing coding sequences for immunoglobulin domains from the parental antibodies mAb 1B12 and panitumumab. The FIT-Ig1 is a hexamer comprised of three component polypeptide chains:

Polypeptide Chain #1 has the domain formula: VL_{1B12}-CL fused directly to VHₚₐₙᵢ-CH1 fused directly to hinge-CH2-CH3 (a human IgG1 Fc region);

Polypeptide Chain #2 has the domain formula: VH_{1B12}-CH1; and

Polypeptide Chain #3 has the domain formula: VLₚₐₙᵢ-CL.

The amino acid sequences for the three expressed FIT-Igl polypeptide chains, including N-terminal signal sequences, are shown in Table 1 below:

TABLE 1
Amino Acid Sequences of FIT-Ig1 Component Polypeptide Chains

| Polypeptide | | |
|---|---|---|
| and | SEQ | Amino Acid Sequence |
| features | ID NO: | 1234567890123456789012345678901234567890 |
| FIT-Ig1 Polypeptide Chain | 4 | MDMRVPAQLLGLLLLWFPGSRCEIVLTQSPATLSLSPGER |
| #1 with N-terminal signal | | ATLSCRASQSVSSYLAWYQQKPGQAPRLLIYDASNRATGI |
| sequence (underlined) | | PARFSGSGSGTDFTLTISSLEPEDFAVYYCQQRSNWPTFG |
| order of specificity: | | QGTKVEIKRTVAAPSVFIFPPSDEQLKSGTASVVCLLNNF |
| PD-L1 (1B12)/ | | YPREAKVQWKVDNALQSGNSQESVTEQDSKDSTYSLSSTL |
| EGFR (panitumumab) | | TLSKADYEKHKVYACEVTHQGLSSPVTKSFNRGECQVQLQ |
| (CDRs in VL and VH | | ESGPGLVKPSETLSLTCTVSGGSVSSGDYYWTWIRQSPGK |
| underlined in bold) | | GLEWIGHIYYSGNTNYNPSLKSRLTISIDTSKTQFSLKLS |
| | | SVTAADTAIYYCVRDRVTGAFDIWGQGTMVTVSSASTKGP |
| | | SVFPLAPSSKSTSGGTAALGCLVKDYFPEPVTVSWNSGAL |
| | | TSGVHTFPAVLQSSGLYSLSSVVTVPSSSLGTQTYICNVN |
| | | HKPSNTKVDKKVEPKSCDKTHTCPPCPAPELLGGPSVFLF |
| | | PPKPKDTLMISRTPEVTCVVVDVSHEDPEVKFNWYVDGVE |
| | | VHNAKTKPREEQYNSTYRVVSVLTVLHQDWLNGKEYKCKV |
| | | SNKALPAPIEKTISKAKGQPREPQVYTLPPSREEMTKNQV |
| | | SLTCLVKGFYPSDIAVEWESNGQPENNYKTTPPVLDSDGS |
| | | FFLYSKLTVDKSRWQQGNVFSCSVMHEALHNHYTQKSLSL |
| | | SPGK |
| signal sequence | 5 | MDMRVPAQLLGLLLLWFPGSRC |
| VL_{1B12}-CL | 6 | EIVLTQSPATLSLSPGERATLSCRASQSVSSYLAWYQQKP |
| (VL_{1B12} underlined) | | GQAPRLLIYDASNRATGIPARFSGSGSGTDFTLTISSLEP |
| | | EDFAVYYCQQRSNWPTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |
| VHₚₐₙᵢ-CH1 | 7 | QVQLQESGPGLVKPSETLSLTCTVSGGSVSSGDYYWTWIR |
| (VHₚₐₙᵢ underlined) | | QSPGKGLEWIGHIYYSGNTNYNPSLKSRLTISIDTSKTQF |
| | | SLKLSSVTAADTAIYYCVRDRVTGAFDIWGQGTMVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSC |
| hinge-CH2-CH3 | 8 | DKTHTCPPCPAPELLGGPSVFLFPPKPKDTLMISRTPEVT |
| of human IgG1 | | CVVVDVSHEDPEVKFNWYVDGVEVHNAKTKPREEQYNSTY |
| | | RVVSVLTVLHQDWLNGKEYKCKVSNKALPAPIEKTISKAK |
| | | GQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| FIT-Ig1 Polypeptide Chain | 9 | MEFGLSWLFLVAILKGVQCQVQLVQSGAEVKKPGSSVKVS |
| #2 with N-terminal signal | | CKTSGDTFSSYAISWVRQAPGQGLEWMGGIIPIFGRAHYA |
| sequence (underlined) | | QKFQGRVTITADESTSTAYMELSSLRSEDTAVYFCARKFH |
| (CDRs underlined in bold) | | FVSGSPFGMDVWGQGTTVTVSSASTKGPSVFPLAPSSKST |
| | | SGGTAALGCLVKDYFPEPVTVSWNSGALTSGVHTFPAVLQ |
| | | SSGLYSLSSVVTVPSSSLGTQTYICNVNHKPSNTKVDKKV |
| | | EPKSC |
| signal sequence | 10 | MEFGLSWLFLVAILKGVQC |
| VH_{1B12}-CH1 | 11 | QVQLVQSGAEVKKPGSSVKVSCKTSGDTFSSYAISWVRQA |
| (VH_{1B12} underlined) | | PGQGLEWMGGIIPIFGRAHYAQKFQGRVTITADESTSTAY |
| | | MELSSLRSEDTAVYFCARKFHFVSGSPFGMDVWGQGTTVT |
| | | VSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSC |
| FIT-Ig1 Polypeptide Chain | 12 | MDMRVPAQLLGLLLLWFPGSRCDIQMTQSPSSLSASVGDR |
| #3 with N-terminal signal | | VTITCQASQDISNYLNWYQQKPGKAPKLLIYDASNLETGV |
| sequence (underlined) | | PSRFSGSGSGTDFTFTISSLQPEDIATYFCQHFDHLPLAF |
| (CDRs underlined in bold) | | GGGTKVEIKRTVAAPSVFIFPPSDEQLKSGTASVVCLLNN |
| | | FYPREAKVQWKVDNALQSGNSQESVTEQDSKDSTYSLSST |
| | | LTLSKADYEKHKVYACEVTHQGLSSPVTKSFNRGEC |
| signal sequence | 5 | MDMRVPAQLLGLLLLWFPGSRC |
| VLₚₐₙi-CL | 13 | DIQMTQSPSSLSASVGDRVTITCQASQDISNYLNWYQQKP |
| (VLₚₐₙᵢ underlined) | | GKAPKLLIYDASNLETGVPSRFSGSGSGTDFTFTISSLQP |
| | | EDIATYFCQHFDHLPLAFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

### Example 1.2: FIT-Ig2

An EGFR/PD-L1 FIT-Ig designated “FIT-Ig2” (also referred to as “EGFR/PD-L1 FIT-Ig2”) was constructed utilizing coding sequences for immunoglobulin domains from the parental antibodies panitumumab and mAb 1B12. The FIT-Ig2 is a hexamer comprised of three component polypeptide chains:

Polypeptide Chain #1 has the domain formula: VLₚₐₙᵢ-CL fused directly to VH_{1B12}-CH1 fused directly to hinge-CH2-CH3 (a human IgG1 Fc region);

Polypeptide Chain #2 has the domain formula: VHₚₐₙᵢ-CH1; and

Polypeptide chain #3 has the domain formula: VL_{1B12}-CL.

The amino acid sequences for the three expressed FIT-Ig2 polypeptide chains, including N-terminal signal sequences, are shown in Table 2 below:

TABLE 2
Amino Acid Sequences of FIT-Ig2 Component Polypeptide Chains

| Polypeptide | | |
|---|---|---|
| and | SEQ | Amino Acid Sequence |
| features | ID NO: | 1234567890123456789012345678901234567890 |
| FIT-Ig2 Polypeptide Chain | 14 | MDMRVPAQLLGLLLLWFPGSRCDIQMTQSPSSLSASVGDR |
| #1 with N-terminal signal | | VTITCQASQDISNYLNWYQQKPGKAPKLLIYDASNLETGV |
| sequence (underlined) | | PSRFSGSGSGTDFTFTISSLQPEDIATYFCQHFDHLPLAF |
| Order of specificity: | | GGGTKVEIKRTVAAPSVFIFPPSDEQLKSGTASVVCLLNN |
| EGFR (panitumumab)/ | | FYPREAKVQWKVDNALQSGNSQESVTEQDSKDSTYSLSST |
| PD-L1 (1B12) | | LTLSKADYEKHKVYACEVTHQGLSSPVTKSFNRGECQVQL |
| (CDRs in VL and VH | | VQSGAEVKKPGSSVKVSCKTSGDTFSSYAISWVRQAPGQG |
| underlined in bold) | | LEWMGGIIPIFGRAHYAQKFQGRVTITADESTSTAYMELS |
| | | SLRSEDTAVYFCARKFHFVSGSPFGMDVWGQGTTVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSCDKTHTCPPCPAPELLGGP |
| | | SVFLFPPKPKDTLMISRTPEVTCVVVDVSHEDPEVKFNWY |
| | | VDGVEVHNAKTKPREEQYNSTYRVVSVLTVLHQDWLNGKE |
| | | YKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYTQ |
| | | KSLSLSPGK |
| signal sequence | 5 | MDMRVPAQLLGLLLLWFPGSRC |
| VLₚₐₙᵢ-CL | 15 | DIQMTQSPSSLSASVGDRVTITCQASQDISNYLNWYQQKP |
| (VL underlined) | | GKAPKLLIYDASNLETGVPSRFSGSGSGTDFTFTISSLQP |
| | | EDIATYFCQHFDHLPLAFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| VH_{1B12}-CH1 | 16 | QVQLVQSGAEVKKPGSSVKVSCKTSGDTFSSYAISWVRQA |
| (VH underlined) | | PGQGLEWMGGIIPIFGRAHYAQKFQGRVTITADESTSTAY |
| | | MELSSLRSEDTAVYFCARKFHFVSGSPFGMDVWGQGTTVT |
| | | VSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSC |
| hinge-CH2-CH3 | 8 | DKTHTCPPCPAPELLGGPSVFLFPPKPKDTLMISRTPEVT |
| of human IgG1 | | CVVVDVSHEDPEVKFNWYVDGVEVHNAKTKPREEQYNSTY |
| | | RVVSVLTVLHQDWLNGKEYKCKVSNKALPAPIEKTISKAK |
| | | GQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| FIT-Ig2 Polypeptide Chain | 17 | MEFGLSWLFLVAILKGVQCQVQLQESGPGLVKPSETLSLT |
| #2 with N-terminal signal | | CTVSGGSVSSGDYYWTWIRQSPGKGLEWIGHIYYSGNTNY |
| sequence (underlined) | | NPSLKSRLTISIDTSKTQFSLKLSSVTAADTAIYYCVRDR |
| (CDRs underlined in bold) | | VTGAFDIWGQGTMVTVSSASTKGPSVFPLAPSSKSTSGGT |
| | | AALGCLVKDYFPEPVTVSWNSGALTSGVHTFPAVLQSSGL |
| | | YSLSSVVTVPSSSLGTQTYICNVNHKPSNTKVDKKVEPKS |
| | | C |
| signal sequence | 10 | MEFGLSWLFLVAILKGVQC |
| VHₚₐₙᵢ-CH1 | 18 | QVQLQESGPGLVKPSETLSLTCTVSGGSVSSGDYYWTWIR |
| (VHₚₐₙᵢ underlined) | | QSPGKGLEWIGHIYYSGNTNYNPSLKSRLTISIDTSKTQF |
| | | SLKLSSVTAADTAIYYCVRDRVTGAFDIWGQGTMVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | CNVNHKPSNTKVDKKVEPKSC |
| FIT-Ig2 Polypeptide Chain | 19 | MDMRVPAQLLGLLLLWFPGSRCEIVLTQSPATLSLSPGER |
| #3 with N-terminal signal | | ATLSCRASQSVSSYLAWYQQKPGQAPRLLIYDASNRATGI |
| sequence | | PARESGSGSGTDFTLTISSLEPEDFAVYYCQQRSNWPTFG |
| (CDRs underlined in bold) | | QGTKVEIKRTVAAPSVFIFPPSDEQLKSGTASVVCLLNNF |
| | | YPREAKVQWKVDNALQSGNSQESVTEQDSKDSTYSLSSTL |
| | | TLSKADYEKHKVYACEVTHQGLSSPVTKSFNRGEC |
| signal sequence | 5 | MDMRVPAQLLGLLLLWFPGSRC |
| VL_{1B12}-CL | 20 | EIVLTQSPATLSLSPGERATLSCRASQSVSSYLAWYQQKP |
| (VL_{1B12} underlined) | | GQAPRLLIYDASNRATGIPARFSGSGSGTDFTLTISSLEP |
| | | EDFAVYYCQQRSNWPTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |

### Example 1.3: FIT-Ig3

A PD-L1/EGFR FIT-Ig designated “FIT-Ig3” (also referred to as “PD-L1/EGFR FIT-Ig3”) was constructed utilizing coding sequences for immunoglobulin domains from the parental antibodies 10A5 and panitumumab. The FIT-Ig3 is a hexamer comprised of three component polypeptide chains:

Polypeptide Chain #1 has the domain formula: VL_{10A5}-CL fused directly to VHₚₐₙᵢ-CH1 fused directly to hinge-CH2-CH3 (a human IgG1 Fc region);

Polypeptide Chain #2 has the domain formula: VH_{10A5}-CH1; and

Polypeptide Chain #3 has the domain formula: VLₚₐₙᵢ-CL.

The amino acid sequences for the three expressed FIT-Ig3 polypeptide chains, including N-terminal signal sequences, are shown in Table 3 below:

TABLE 3
Amino Acid Sequences of FIT-Ig3 Component Polypeptide Chains

| Polypeptide | | |
|---|---|---|
| and | SEQ | Amino Acid Sequence |
| features | ID NO: | 1234567890123456789012345678901234567890 |
| FIT-Ig3 Polypeptide Chain | 21 | MDMRVPAQLLGLLLLWFPGSRCDIQMTQSPSSLSASVGDR |
| #1 with N-terminal signal | | VTITCRASQGISSWLAWYQQKPEKAPKSLIYAASSLQSGV |
| sequence (underlined) | | PSRFSGSGSGTDFTLTISSLQPEDFATYYCQQYNSYPYTF |
| order of specificity: | | GQGTKLEIKRTVAAPSVFIFPPSDEQLKSGTASVVCLLNN |
| PD-L1 (10A5)/EGFR | | FYPREAKVQWKVDNALQSGNSQESVTEQDSKDSTYSLSST |
| (panitumumab) | | LTLSKADYEKHKVYACEVTHQGLSSPVTKSFNRGECQVQL |
| (CDRs in VL and VH | | QESGPGLVKPSETLSLTCTVSGGSVSSGDYYWTWIRQSPG |
| underlined in bold) | | KGLEWIGHIYYSGNTNYNPSLKSRLTISIDTSKTQFSLKL |
| | | SSVTAADTAIYYCVRDRVTGAFDIWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCDKTHTCPPCPAPELLGGPSVFL |
| | | FPPKPKDTLMISRTPEVTCVVVDVSHEDPEVKFNWYVDGV |
| | | EVHNAKTKPREEQYNSTYRVVSVLTVLHQDWLNGKEYKCK |
| | | VSNKALPAPIEKTISKAKGQPREPQVYTLPPSREEMTKNQ |
| | | VSLTCLVKGFYPSDIAVEWESNGQPENNYKTTPPVLDSDG |
| | | SFFLYSKLTVDKSRWQQGNVFSCSVMHEALHNHYTQKSLS |
| | | LSPGK |
| signal sequence | 5 | MDMRVPAQLLGLLLLWFPGSRC |
| VL_{10A5}-CL | 22 | DIQMTQSPSSLSASVGDRVTITCRASQGISSWLAWYQQKP |
| (VL underlined) | | EKAPKSLIYAASSLQSGVPSRFSGSGSGTDFTLTISSLQP |
| | | EDFATYYCQQYNSYPYTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| VHₚₐₙᵢ-CH1 | 23 | QVQLQESGPGLVKPSETLSLTCTVSGGSVSSGDYYWTWIR |
| (VH underlined) | | QSPGKGLEWIGHIYYSGNTNYNPSLKSRLTISIDTSKTQF |
| | | SLKLSSVTAADTAIYYCVRDRVTGAFDIWGQGTMVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSC |
| hinge-CH2-CH3 | 8 | DKTHTCPPCPAPELLGGPSVFLFPPKPKDTLMISRTPEVT |
| of human IgG1 | | CVVVDVSHEDPEVKFNWYVDGVEVHNAKTKPREEQYNSTY |
| | | RVVSVLTVLHQDWLNGKEYKCKVSNKALPAPIEKTISKAK |
| | | GQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| FIT-Ig3 Polypeptide Chain | 24 | MEFGLSWLELVAILKGVQCQVQLVQSGAEVKKPGASVKVS |
| #2 with N-terminal signal | | CKASGYTFTSYDVHWVRQAPGQRLEWMGWLHADTGITKFS |
| sequence (underlined) | | QKFQGRVTITRDTSASTAYMELSSLRSEDTAVYYCARERI |
| (CDRs underlined in bold) | | QLWFDYWGQGTLVTVSSASTKGPSVFPLAPSSKSTSGGTA |
| | | ALGCLVKDYFPEPVTVSWNSGALTSGVHTFPAVLQSSGLY |
| | | SLSSVVTVPSSSLGTQTYICNVNHKPSNTKVDKKVEPKSC |
| signal sequence | 10 | MEFGLSWLFLVAILKGVQC |
| VH_{10A5}-CH1 | 25 | QVQLVQSGAEVKKPGASVKVSCKASGYTFTSYDVHWVRQA |
| (VH underlined) | | PGQRLEWMGWLHADTGITKFSQKFQGRVTITRDTSASTAY |
| | | MELSSLRSEDTAVYYCARERIQLWFDYWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSC |
| FIT-Ig3 Polypeptide Chain | 26 | MDMRVPAQLLGLLLLWFPGSRCDIQMTQSPSSLSASVGDR |
| #3 with N-terminal signal | | VTITCQASQDISNYLNWYQQKPGKAPKLLIYDASNLETGV |
| sequence (underlined) | | PSRFSGSGSGTDFTFTISSLQPEDIATYFCQHFDHLPLAF |
| (CDRs underlined in bold) | | GGGTKVEIKRTVAAPSVFIFPPSDEQLKSGTASVVCLLNN |
| | | FYPREAKVQWKVDNALQSGNSQESVTEQDSKDSTYSLSST |
| | | LTLSKADYEKHKVYACEVTHQGLSSPVTKSFNRGEC |
| signal sequence | 5 | MDMRVPAQLLGLLLLWFPGSRC |
| VLₚₐₙᵢ-CL | 27 | DIQMTQSPSSLSASVGDRVTITCQASQDISNYLNWYQQKP |
| (VL underlined) | | GKAPKLLIYDASNLETGVPSRFSGSGSGTDFTFTISSLQP |
| | | EDIATYFCQHFDHLPLAFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

### Example 1.4: FIT-Ig4

An EGFR/PD-L1 FIT-Ig designated “FIT-Ig4” (also referred to as “EGFR/PD-L1 FIT-Ig4”) was constructed utilizing coding sequences for immunoglobulin domains from the parental antibodies panitumumab and mAb 10A5. The FIT-Ig4 is a hexamer comprised of three component polypeptide chains:

Polypeptide Chain #1 has the domain formula: VLₚₐₙᵢ-CL fused directly to VH_{10A5}-CH1 fused directly to hinge-CH2-CH3 (a human IgG1 Fc region);

Polypeptide Chain #2 has the domain formula: VHₚₐₙᵢ-CH1; and

Polypeptide Chain #3 has the domain formula: VL_{10A5}-CL.

The amino acid sequences for the three expressed FIT-Ig4 polypeptide chains, including N-terminal signal sequences, are shown in Table 4 below:

TABLE 4
Amino Acid Sequences of FIT-Ig4 Component Polypeptide Chains

| | SEQ | Amino Acid Sequence |
|---|---|---|
| Polypeptide | ID NO: | 1234567890123456789012345678901234567890 |
| FIT-Ig4 Polypeptide Chain | 28 | MDMRVPAQLLGLLLLWFPGSRCDIQMTQSPSSLSASVGDR |
| #1 with N-terminal signal | | VTITCQASQDISNYLNWYQQKPGKAPKLLIYDASNLETGV |
| sequence (underlined) | | PSRFSGSGSGTDFTFTISSLQPEDIATYFCQHFDHLPLAF |
| order of specificity: | | GGGTKVEIKRTVAAPSVFIFPPSDEQLKSGTASVVCLLNN |
| EGFR (panitumumab)/ | | FYPREAKVQWKVDNALQSGNSQESVTEQDSKDSTYSLSST |
| PD-L1 (10A5) | | LTLSKADYEKHKVYACEVTHQGLSSPVTKSFNRGECQVQL |
| (CDRs in VL and VH | | VQSGAEVKKPGASVKVSCKASGYTFTSYDVHWVRQAPGQR |
| underlined in bold) | | LEWMGWLHADTGITKFSQKFQGRVTITRDTSASTAYMELS |
| | | SLRSEDTAVYYCARERIQLWFDYWGQGTLVTVSSASTKGP |
| | | SVFPLAPSSKSTSGGTAALGCLVKDYFPEPVTVSWNSGAL |
| | | TSGVHTFPAVLQSSGLYSLSSVVTVPSSSLGTQTYICNVN |
| | | HKPSNTKVDKKVEPKSCDKTHTCPPCPAPELLGGPSVFLF |
| | | PPKPKDTLMISRTPEVTCVVVDVSHEDPEVKFNWYVDGVE |
| | | VHNAKTKPREEQYNSTYRVVSVLTVLHQDWLNGKEYKCKV |
| | | SNKALPAPIEKTISKAKGQPREPQVYTLPPSREEMTKNQV |
| | | SLTCLVKGFYPSDIAVEWESNGQPENNYKTTPPVLDSDGS |
| | | FFLYSKLTVDKSRWQQGNVFSCSVMHEALHNHYTQKSLSL |
| | | SPGK |
| signal sequence | 5 | MDMRVPAQLLGLLLLWFPGSRC |
| VLₚₐₙᵢ-CL | 29 | DIQMTQSPSSLSASVGDRVTITCQASQDISNYLNWYQQKP |
| (VL underlined) | | GKAPKLLIYDASNLETGVPSRFSGSGSGTDFTFTISSLQP |
| | | EDIATYFCQHFDHLPLAFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| VH_{10A5}-CH1 | 30 | QVQLVQSGAEVKKPGASVKVSCKASGYTFTSYDVHWVRQA |
| (VH underlined) | | PGQRLEWMGWLHADTGITKFSQKFQGRVTITRDTSASTAY |
| | | MELSSLRSEDTAVYYCARERIQLWFDYWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSC |
| hinge-CH2-CH3 | 8 | DKTHTCPPCPAPELLGGPSVFLFPPKPKDTLMISRTPEVT |
| of human IgG1 | | CVVVDVSHEDPEVKFNWYVDGVEVHNAKTKPREEQYNSTY |
| | | RVVSVLTVLHQDWLNGKEYKCKVSNKALPAPIEKTISKAK |
| | | GQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| FIT-Ig4 Polypeptide Chain | 31 | MEFGLSWLFLVAILKGVQCQVQLQESGPGLVKPSETLSLT |
| #2 with N-terminal signal | | CTVSGGSVSSGDYYWTWIRQSPGKGLEWIGHIYYSGNTNY |
| sequence | | NPSLKSRLTISIDTSKTQFSLKLSSVTAADTAIYYCVRDR |
| (CDRs underlined in bold) | | VTGAFDIWGQGTMVTVSSASTKGPSVFPLAPSSKSTSGGT |
| | | AALGCLVKDYFPEPVTVSWNSGALTSGVHTFPAVLQSSGL |
| | | YSLSSVVTVPSSSLGTQTYICNVNHKPSNTKVDKKVEPKS |
| | | C |
| signal sequence | 10 | MEFGLSWLFLVAILKGVQC |
| VHₚₐₙᵢ-CH1 | 32 | QVQLQESGPGLVKPSETLSLTCTVSGGSVSSGDYYWTWIR |
| (VH underlined) | | QSPGKGLEWIGHIYYSGNTNYNPSLKSRLTISIDTSKTQF |
| | | SLKLSSVTAADTAIYYCVRDRVTGAFDIWGQGTMVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSC |
| FIT-Ig4 Polypeptide Chain | 33 | MDMRVPAQLLGLLLLWFPGSRCDIQMTQSPSSLSASVGDR |
| #3 with N-terminal signal | | VTITCRASQGISSWLAWYQQKPEKAPKSLIYAASSLQSGV |
| sequence | | PSRFSGSGSGTDFTLTISSLQPEDFATYYCQQYNSYPYTF |
| (CDRs underlined in bold) | | GQGTKLEIKRTVAAPSVFIFPPSDEQLKSGTASVVCLLNN |
| | | FYPREAKVQWKVDNALQSGNSQESVTEQDSKDSTYSLSST |
| | | LTLSKADYEKHKVYACEVTHQGLSSPVTKSFNRGEC |
| signal sequence | 5 | MDMRVPAQLLGLLLLWFPGSRC |
| VL_{10A5}-CL | 34 | DIQMTQSPSSLSASVGDRVTITCRASQGISSWLAWYQQKP |
| (VL underlined) | | EKAPKSLIYAASSLQSGVPSRFSGSGSGTDFTLTISSLQP |
| | | EDFATYYCQQYNSYPYTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

### Example 1.5: FIT-Ig5

A PD-L1/EGFR FIT-Ig designated “FIT-Ig5” (also referred to as “PD-L1/EGFR FIT-Ig5”) was constructed utilizing coding sequences for immunoglobulin domains from the parental antibodies mAb 3G10 and panitumumab. The FIT-Ig5 is a hexamer comprised of three component polypeptide chains:

Polypeptide Chain #1 has the domain formula: VL_{3G10}-CL fused directly to VHₚₐₙᵢ-CH1 fused directly to hinge-CH2-CH3 (a human IgG1 Fc region);

Polypeptide Chain #2 has the domain formula: VH_{3G10}-CH1; and

Polypeptide Chain #3 has the domain formula: VLpani-CL.

The amino acid sequences for the three expressed FIT-Ig5 polypeptide chains, including N-terminal signal sequences, are shown in Table 5 below:

TABLE 5
Amino Acid Sequences of FIT-Ig5 Component Polypeptide Chains

| | SEQ | Amino Acid Sequence |
|---|---|---|
| Polypeptide | ID NO: | 1234567890123456789012345678901234567890 |
| FIT-Ig5 Polypeptide Chain | 35 | MDMRVPAQLLGLLLLWFPGSRCEIVLTQSPATLSLSPGER |
| #1 with N-terminal signal | | ATLSCRASQSVSSYLVWYQQKPGQAPRLLIYDASNRATGI |
| sequence (underlined) | | PARFSGSGSGTDFTLTISSLEPEDFAVYYCQQRSNWPRTF |
| order of specificity | | GQGTKVEIKRTVAAPSVFIFPPSDEQLKSGTASVVCLLNN |
| PD-L1 (3G10)/EGFR | | FYPREAKVQWKVDNALQSGNSQESVTEQDSKDSTYSLSST |
| (panitumumab) | | LTLSKADYEKHKVYACEVTHQGLSSPVTKSFNRGECQVQL |
| (CDRs of VL and VH | | QESGPGLVKPSETLSLTCTVSGGSVSSGDYYWTWIRQSPG |
| underlined in bold) | | KGLEWIGHIYYSGNTNYNPSLKSRLTISIDTSKTQFSLKL |
| | | SSVTAADTAIYYCVRDRVTGAFDIWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCDKTHTCPPCPAPELLGGPSVFL |
| | | FPPKPKDTLMISRTPEVTCVVVDVSHEDPEVKFNWYVDGV |
| | | EVHNAKTKPREEQYNSTYRVVSVLTVLHQDWLNGKEYKCK |
| | | VSNKALPAPIEKTISKAKGQPREPQVYTLPPSREEMTKNQ |
| | | VSLTCLVKGFYPSDIAVEWESNGQPENNYKTTPPVLDSDG |
| | | SFFLYSKLTVDKSRWQQGNVFSCSVMHEALHNHYTQKSLS |
| | | LSPGK |
| signal sequence | 5 | MDMRVPAQLLGLLLLWFPGSRC |
| FIT-Ig5 Polypeptide Chain | 36 | EIVLTQSPATLSLSPGERATLSCRASQSVSSYLVWYQQKP |
| #1 without signal sequence | | GQAPRLLIYDASNRATGIPARESGSGSGTDFTLTISSLEP |
| | | EDFAVYYCQQRSNWPRTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECQVQLQESGPGLVKPSETLSLTCTVSG |
| | | GSVSSGDYYWTWIRQSPGKGLEWIGHIYYSGNTNYNPSLK |
| | | SRLTISIDTSKTQFSLKLSSVTAADTAIYYCVRDRVTGAF |
| | | DIWGQGTMVTVSSASTKGPSVFPLAPSSKSTSGGTAALGC |
| | | LVKDYFPEPVTVSWNSGALTSGVHTFPAVLQSSGLYSLSS |
| | | VVTVPSSSLGTQTYICNVNHKPSNTKVDKKVEPKSCDKTH |
| | | TCPPCPAPELLGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPR |
| | | EPQVYTLPPSREEMTKNQVSLTCLVKGFYPSDIAVEWESN |
| | | GQPENNYKTTPPVLDSDGSFFLYSKLTVDKSRWQQGNVFS |
| | | CSVMHEALHNHYTQKSLSLSPGK |
| VL_{3G10}-CL | 37 | EIVLTQSPATLSLSPGERATLSCRASQSVSSYLVWYQQKP |
| (VL underlined) | | GQAPRLLIYDASNRATGIPARFSGSGSGTDFTLTISSLEP |
| | | EDFAVYYCQQRSNWPRTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| VHₚₐₙᵢ-CH1 | 38 | QVQLQESGPGLVKPSETLSLTCTVSGGSVSSGDYYWTWIR |
| (VH underlined) | | QSPGKGLEWIGHIYYSGNTNYNPSLKSRLTISIDTSKTQF |
| | | SLKLSSVTAADTAIYYCVRDRVTGAFDIWGQGTMVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSC |
| hinge-CH2-CH3 | 8 | DKTHTCPPCPAPELLGGPSVFLFPPKPKDTLMISRTPEVT |
| of human IgG1 | | CVVVDVSHEDPEVKFNWYVDGVEVHNAKTKPREEQYNSTY |
| | | RVVSVLTVLHQDWLNGKEYKCKVSNKALPAPIEKTISKAK |
| | | GQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| FIT-Ig5 Polypeptide Chain | 39 | MEFGLSWLELVAILKGVQCQVQLVQSGAEVKKPGASVKVS |
| #2 with N-terminal signal | | CKASGYTFTDYGFSWVRQAPGQGLEWMGWITAYNGNTNYA |
| sequence (underlined) | | QKLQGRVTMTTDTSTSTVYMELRSLRSDDTAVYYCARDYF |
| (CDRs underlined in bold) | | YGMDVWGQGTTVTVSSASTKGPSVFPLAPSSKSTSGGTAA |
| | | LGCLVKDYFPEPVTVSWNSGALTSGVHTFPAVLQSSGLYS |
| | | LSSVVTVPSSSLGTQTYICNVNHKPSNTKVDKKVEPKSC |
| signal sequence | 10 | MEFGLSWLFLVAILKGVQC |
| VH_{3G10}-CH1 | 40 | QVQLVQSGAEVKKPGASVKVSCKASGYTFTDYGFSWVRQA |
| (VH underlined) | | PGQGLEWMGWITAYNGNTNYAQKLQGRVTMTTDTSTSTVY |
| | | MELRSLRSDDTAVYYCARDYFYGMDVWGQGTTVTVSSAST |
| | | KGPSVFPLAPSSKSTSGGTAALGCLVKDYFPEPVTVSWNS |
| | | GALTSGVHTFPAVLQSSGLYSLSSVVTVPSSSLGTQTYIC |
| | | NVNHKPSNTKVDKKVEPKSC |
| FIT-Ig5 Polypeptide Chain | 41 | MDMRVPAQLLGLLLLWFPGSRCDIQMTQSPSSLSASVGDR |
| #3 with N-terminal signal | | VTITCQASQDISNYLNWYQQKPGKAPKLLIYDASNLETGV |
| sequence (underlined) | | PSRFSGSGSGTDFTFTISSLQPEDIATYFCQHFDHLPLAF |
| (CDRs underlined in bold) | | GGGTKVEIKRTVAAPSVFIFPPSDEQLKSGTASVVCLLNN |
| | | FYPREAKVQWKVDNALQSGNSQESVTEQDSKDSTYSLSST |
| | | LTLSKADYEKHKVYACEVTHQGLSSPVTKSFNRGEC |
| signal sequence | 5 | MDMRVPAQLLGLLLLWFPGSRC |
| VLₚₐₙᵢ-CL | 42 | DIQMTQSPSSLSASVGDRVTITCQASQDISNYLNWYQQKP |
| (VL underlined) | | GKAPKLLIYDASNLETGVPSRFSGSGSGTDFTFTISSLQP |
| | | EDIATYFCQHFDHLPLAFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

### Example 1.6: FIT-Ig6

An EGFR/PD-L1 FIT-Ig designated “FIT-Ig6” (also referred to as “EGFR/PD-L1 FIT-Ig6”) was constructed utilizing coding sequences for immunoglobulin domains from the parental antibodies panitumumab and mAb 3G10. The FIT-Ig6 is a hexamer comprised of three component polypeptide chains:

Polypeptide Chain #1 has the domain formula: VLₚₐₙᵢ-CL fused directly to VH_{3G10}-CH1 fused directly to hinge-CH2-CH3 (a human IgG1 Fc region);

Polypeptide Chain #2 has the domain formula: VHₚₐₙᵢ-CH1; and

Polypeptide chain #3 has the domain formula: VL_{3G10}-CL.

The amino acid sequences for the three expressed FIT-Ig6 polypeptide chains, including N-terminal signal sequences, are shown in Table 6 below:

TABLE 6
Amino Acid Sequences of FIT-Ig6 Component Polypeptide Chains

| | SEQ | Amino Acid Sequence |
|---|---|---|
| Polypeptide | ID NO: | 1234567890123456789012345678901234567890 |
| FIT-Ig6 Polypeptide Chain | 43 | MDMRVPAQLLGLLLLWFPGSRCDIQMTQSPSSLSASVGDR |
| #1 with N-terminal signal | | VTITCQASQDISNYLNWYQQKPGKAPKLLIYDASNLETGV |
| sequence (underlined) | | PSRFSGSGSGTDFTFTISSLQPEDIATYFCQHFDHLPLAF |
| order of specificity | | GGGTKVEIKRTVAAPSVFIFPPSDEQLKSGTASVVCLLNN |
| EGFR (panitumumab)/ | | FYPREAKVQWKVDNALQSGNSQESVTEQDSKDSTYSLSST |
| PD-L1 (3G10) | | LTLSKADYEKHKVYACEVTHQGLSSPVTKSFNRGECQVQL |
| (CDRs of VL and VH | | VQSGAEVKKPGASVKVSCKASGYTFTDYGFSWVRQAPGQG |
| underlined in bold) | | LEWMGWITAYNGNTNYAQKLQGRVTMTTDTSTSTVYMELR |
| | | SLRSDDTAVYYCARDYFYGMDVWGQGTTVTVSSASTKGPS |
| | | VFPLAPSSKSTSGGTAALGCLVKDYFPEPVTVSWNSGALT |
| | | SGVHTFPAVLQSSGLYSLSSVVTVPSSSLGTQTYICNVNH |
| | | KPSNTKVDKKVEPKSCDKTHTCPPCPAPELLGGPSVFLFP |
| | | PKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSREEMTKNQVS |
| | | LTCLVKGFYPSDIAVEWESNGQPENNYKTTPPVLDSDGSF |
| | | FLYSKLTVDKSRWQQGNVFSCSVMHEALHNHYTQKSLSLS |
| | | PGK |
| signal sequence | 5 | MDMRVPAQLLGLLLLWFPGSRC |
| FIT-Ig6 Polypeptide Chain | 1 | DIQMTQSPSSLSASVGDRVTITCQASQDISNYLNWYQQKP |
| #1 without signal sequence | | GKAPKLLIYDASNLETGVPSRFSGSGSGTDFTFTISSLQP |
| | | EDIATYFCQHFDHLPLAFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECQVQLVQSGAEVKKPGASVKVSCKASG |
| | | YTFTDYGFSWVRQAPGQGLEWMGWITAYNGNTNYAQKLQG |
| | | RVTMTTDTSTSTVYMELRSLRSDDTAVYYCARDYFYGMDV |
| | | WGQGTTVTVSSASTKGPSVFPLAPSSKSTSGGTAALGCLV |
| | | KDYFPEPVTVSWNSGALTSGVHTFPAVLQSSGLYSLSSVV |
| | | TVPSSSLGTQTYICNVNHKPSNTKVDKKVEPKSCDKTHTC |
| | | PPCPAPELLGGPSVFLFPPKPKDTLMISRTPEVTCVVVDV |
| | | SHEDPEVKFNWYVDGVEVHNAKTKPREEQYNSTYRVVSVL |
| | | TVLHQDWLNGKEYKCKVSNKALPAPIEKTISKAKGQPREP |
| | | QVYTLPPSREEMTKNQVSLTCLVKGFYPSDIAVEWESNGQ |
| | | PENNYKTTPPVLDSDGSFFLYSKLTVDKSRWQQGNVFSCS |
| | | VMHEALHNHYTQKSLSLSPGK |
| VLₚₐₙᵢ-CL | 44 | DIQMTQSPSSLSASVGDRVTITCQASQDISNYLNWYQQKP |
| (VL underlined) | | GKAPKLLIYDASNLETGVPSRFSGSGSGTDFTFTISSLQP |
| | | EDIATYFCQHFDHLPLAFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| VH_{3G10}-CH1 | 45 | QVQLVQSGAEVKKPGASVKVSCKASGYTFTDYGFSWVRQA |
| (VH underlined) | | PGQGLEWMGWITAYNGNTNYAQKLQGRVTMTTDTSTSTVY |
| | | MELRSLRSDDTAVYYCARDYFYGMDVWGQGTTVTVSSAST |
| | | KGPSVFPLAPSSKSTSGGTAALGCLVKDYFPEPVTVSWNS |
| | | GALTSGVHTFPAVLQSSGLYSLSSVVTVPSSSLGTQTYIC |
| | | NVNHKPSNTKVDKKVEPKSC |
| hinge-CH2-CH3 | 8 | DKTHTCPPCPAPELLGGPSVFLFPPKPKDTLMISRTPEVT |
| of human IgG1 | | CVVVDVSHEDPEVKFNWYVDGVEVHNAKTKPREEQYNSTY |
| | | RVVSVLTVLHQDWLNGKEYKCKVSNKALPAPIEKTISKAK |
| | | GQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| FIT-Ig6 Polypeptide Chain | 46 | MEFGLSWLFLVAILKGVQCQVQLQESGPGLVKPSETLSLT |
| #2 with N-terminal signal | | CTVSGGSVSSGDYYWTWIRQSPGKGLEWIGHIYYSGNTNY |
| sequence (underlined) | | NPSLKSRLTISIDTSKTQFSLKLSSVTAADTAIYYCVRDR |
| (CDRs underlined in bold) | | VTGAFDIWGQGTMVTVSSASTKGPSVFPLAPSSKSTSGGT |
| | | AALGCLVKDYFPEPVTVSWNSGALTSGVHTFPAVLQSSGL |
| | | YSLSSVVTVPSSSLGTQTYICNVNHKPSNTKVDKKVEPKS |
| | | C |
| signal sequence | 10 | MEFGLSWLFLVAILKGVQC |
| VHₚₐₙᵢ-CH1 | 2 | QVQLQESGPGLVKPSETLSLTCTVSGGSVSSGDYYWTWIR |
| (VH underlined) | | QSPGKGLEWIGHIYYSGNTNYNPSLKSRLTISIDTSKTQF |
| | | SLKLSSVTAADTAIYYCVRDRVTGAFDIWGQGTMVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSC |
| FIT-Ig6 Polypeptide Chain | 47 | MDMRVPAQLLGLLLLWFPGSRCEIVLTQSPATLSLSPGER |
| #3 with N-terminal signal | | ATLSCRASQSVSSYLVWYQQKPGQAPRLLIYDASNRATGI |
| sequence (underlined) | | PARFSGSGSGTDFTLTISSLEPEDFAVYYCQQRSNWPRTF |
| (CDRs underlined in bold) | | GQGTKVEIKRTVAAPSVFIFPPSDEQLKSGTASVVCLLNN |
| | | FYPREAKVQWKVDNALQSGNSQESVTEQDSKDSTYSLSST |
| | | LTLSKADYEKHKVYACEVTHQGLSSPVTKSFNRGEC |
| signal sequence | 5 | MDMRVPAQLLGLLLLWFPGSRC |
| VL_{3G10}-CL | 3 | EIVLTQSPATLSLSPGERATLSCRASQSVSSYLVWYQQKP |
| (VL underlined) | | GQAPRLLIYDASNRATGIPARESGSGSGTDFTLTISSLEP |
| | | EDFAVYYCQQRSNWPRTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

### Example 1.7: Expression of FIT-Ig Binding Proteins

The six FIT-Ig constructs FIT-Ig1, FIT-Ig2, FIT-Ig3, FIT-Ig4, FIT-Ig5, FIT-Ig6 are a type of bispecific, multivalent binding protein known as a no-linker Fabs-in-Tandem Immunoglobulin (or no-linker FIT-Ig) described generally in WO 2015/103072 and WO 2017/136820. The binding proteins were produced by co-expression of three component polypeptide chains in a mammalian host cell transfected with expression vectors for all three chains. The design of the binding proteins calls for a first polypeptide chain (or “heavy chain”) to pair with a second polypeptide chain (or “first light chain”) and a third polypeptide chain (or “second light chain”), to form functional tandem Fab moieties, and also the heavy chain is designed to dimerize via the Fc region (hinge-CH2-CH3), such that a six-chain binding protein exhibiting four intact Fab binding sites is formed. No synthetic amino acid linker peptides are used to connect immunoglobulin domains, hence the designation “no-linker FIT-Igs”; such binding proteins were found to express well in host cells, similarly to recombinantly produced monoclonal antibodies, and the absence of linkers prevented introduction of possible immunogenic sites, which possibly leads to more rapid clearance of FIT-Igs featuring linkers. The no-linker FIT-Igs also were discovered to exhibit binding properties for their target antigens that were comparable to the parental antibodies on which the VH-CH1 and VL-CL were based, surprisingly avoiding the steric hindrance that had been found between “inner” and “outer” binding sites is previous engineered antibodies having tandemly arranged antigen binding sites. However, as shown herein, despite using the no-linker FIT-Ig model, prior constructs of FIT-Ig proteins that bind PD-L1 and EGFR, such as FIT-Igs 1-5, exhibited uncharacteristically low levels of expression and/or undesirably high percentages of aggregates to efficiently be used in the routine pre-clinical and clinical assays required for assessment as a therapeutic anti-cancer drug.

In the binding proteins FIT-Ig1, FIT-Ig3, and Fit-Ig5, the N-terminal or “outer” Fab binding sites bind PD-L1 and the adjacent “inner” Fab binding sites bind EGFR. The outer Fab fragment (of anti-PD-L1 mAb 1B12, 10A5, or 3G10) is joined to the inner Fab fragment (of anti-EGFR panitumumab) only through the heavy chain by direct fusion of VL-CL (of anti-PD-L1 mAb 1B12, 10A5, or 3G10) at its C-terminus to the N-terminus of VH-CH1 (of anti-EGFR panitumumab), without the use of linkers connecting the immunoglobulin domains.

In the binding proteins FIT-Ig2, FIT-Ig4, and Fit-Ig6, the N-terminal or “outer” Fab binding sites bind EGFR and the adjacent “inner” Fab binding sites bind PD-L1. The outer Fab fragment (of anti-EGFR panitumumab) is joined to the inner Fab fragment (of anti-PD-L1 mAb 1B12, 10A5, or 3G10) only through the heavy chain by direct fusion of VL-CL (of anti-EGFR panitumumab) at its C-terminus to the N-terminus of VH-CH1 (of anti-PD-L1 mAbs 1B12, 10A5, or 3G10)), without the use of linkers connecting the immunoglobulin domains.

Each of the FIT-Igs were transiently expressed using transfected human embryonic kidney 293E (HEK293) cells. The HEK293E cell line is a derivative of HEK293 that expresses EBNA-1 and provides enhanced levels of expression of vector-encoded recombinant proteins.

The expression vectors permit the expression of each of three polypeptide chains for any FIT-Ig binding protein in which the first (heavy) polypeptide chain has the structural formula: VL_{A}-CL-VH_{B}-CH1-Fc, the second (first light) polypeptide chain has the structural formula: VH_{A}-CH1, and the third (second light) polypeptide chain has the structural formula: VL_{B}-CL, wherein VL_{A} and VH_{A} are variable domains of the antigen binding site of a first parental antibody, and VL_{B} and VH_{B} are the variable domains of the antigen binding site of a second parental antibody.

As illustrated in FIG. 1, in order to express the first polypeptide chain (“Heavy Chain”) of a FIT-Ig binding protein, a DNA molecule was synthesized (“DNA Synthesis”) that encoded the VL_{A}-CL-VH_{B} segment of the first polypeptide chain The DNA molecule was then inserted into the multiple cloning site (MCS) of a pcDNA3.1 expression vector using homologous recombination in cells of Escherichia coli. This homologous recombination method relies on the principle that recombinase positive E. coli cells are able to recombine homologous sequences with a high rate of specificity and speed. Linear DNA fragments comprising a coding sequence of interest are generated by polymerase chain reaction (PCR) to include sequences on the 5′ and 3′ ends that are homologous with end sequences on a linearized vector. When the PCR product and linear vector are mixed and transformed into competent E. coli cells, endogenous bacterial recombinase activity is able to join the two DNA fragments resulting in a circular plasmid. Then the inserted DNA molecule was positioned downstream of the vector's strong cytomegalovirus (CMV)-enhancer promoter, downstream and in frame of a DNA segment encoding an amino terminal signal peptide (SP), and upstream and in frame with an inserted DNA molecule encoding an antibody CH1 domain linked to an antibody Fc region comprising hinge region-CH2-CH3 domains (designated “h-CH2-CH3” in FIG. 1).

As also illustrated in FIG. 1, in order to express the second polypeptide chain (Light Chain #1) of a FIT-Ig binding protein, a DNA segment was synthesized that encoded an antibody VH_{A} domain, which was then inserted into the multiple cloning site (MCS) of a pcDNA3.1 expression vector so that the inserted DNA molecule was positioned downstream of the vector's strong CMV-enhancer promoter, downstream and in frame of a DNA segment encoding an amino terminal signal peptide (SP), and upstream and in frame with an inserted DNA molecule encoding an antibody CH1 domain.

As illustrated in FIG. 1, in order to express the third polypeptide chain (Light Chain #3) of a FIT-Ig binding protein, a DNA segment was synthesized that encoded an antibody VL_{B} domain, which was then inserted into the multiple cloning site (MCS) of a pcDNA3.1 expression vector so that the inserted DNA molecule was positioned downstream of the vector's strong CMV-enhancer promoter, downstream and in frame of a DNA segment encoding an amino terminal signal peptide (SP), and upstream and in frame with an inserted DNA molecule encoding an antibody CL domain.

Sequences of the resulting expression vectors were confirmed by DNA sequencing.

The resulting expression vectors coding for the three component polypeptide chains of each of the FIT-Igs were transfected into HEK293E cells using a molar ratio for heavy chain:light chain #1:light chain #2 of 1:3:3. This was designed to cause proportionally more of light chains #1 and #2 to be expressed relative to the heavy chain, which in turn would decrease the occurrence of VL-CL and VH-CH1 segments on the heavy chain that were not paired with corresponding light chains, and thus would fail to form a functional Fab fragment. See, WO 2015/103072. The HEK293E cells were transfected with the expression vectors using polyethyleneimine (PEI) as a transfection agent. In this transfection protocol, the expression vectors in FreeStyle™ 293 Expression Medium were mixed with the PEI with the final concentration of DNA to PEI ratio of 1:2, incubated for 15-20 minutes at room temperature, and then added to the HEK293E cells (1.0-1.2×10⁶/ml, cell viability>95%) at 60 μg DNA/120 ml culture. After 6-24 hours of culture in shaker, peptone was added to the transfected cells at a final concentration of 5%, with shaking at 125 rpm/min., at 37° C., 8% CO₂. On the 6th-7th day, supernatant was harvested by centrifugation and filtration, and FIT-Ig protein was purified using Protein A chromatography (GE healthcare, US) according to the manufacturer's instructions. The proteins were analyzed by SDS-PAGE and their concentrations determined by UV absorbance at 280 nm and bicinchoninic acid protein assay (BCA) (Pierce BCA Protein Assay Kit, Thermo Fisher Scientific).

FIT-Ig protein expression products were purified by Protein A chromatography. The composition and purity of the purified FIT-Igs were then analyzed by size exclusion chromatography (SEC). Purified FIT-Ig, in PBS, was applied on a TSKgel SuperSW3000, 300×4.6 mm column (TOSOH). An HPLC instrument, Model U3000 (DIONEX) was used for SEC using UV detection at 280 nm and 214 nm. Species detected by SEC other than the six-polypeptide chain FIT-Ig6 monomer (molecular weight of 240,000 daltons), including larger (higher molecular weight) aggregates and smaller species, including fragments of the FIT-Ig6 monomer are impurities.

The SEC elution profile for each of FIT-Ig1 through FIT-Ig6 is shown, respectively, in FIGS. 2-7.

The SEC elution profile for FIT-Ig1 shown in FIG. 2 revealed multiple overlapping peaks of protein aggregates. The profile was too complex to permit detailed analysis of individual peak species.

The SEC elution profile for FIT-Ig2 shown in FIG. 3 revealed a major peak at the position expected for the six-polypeptide FIT-Ig2 monomer preceded by at least two additional peaks of protein aggregates.

The SEC elution profile for FIT-Ig3 shown in FIG. 4 revealed multiple overlapping peaks of protein aggregates. The profile was too complex to permit detailed analysis of individual peak species.

The SEC elution profile for FIT-Ig4 shown in FIG. 5 revealed a major peak at the position expected for the six-polypeptide FIT-Ig4 monomer preceded by a minor peak of protein aggregates and followed by another minor peak of unknown species (possibly a degradation product).

The SEC elution profile for FIT-Ig5 shown in FIG. 6 revealed a major peak at the position expected for the six-polypeptide FIT-Ig5 monomer preceded by a minor peak of protein aggregates that was significantly smaller than any found in the previous profiles in FIGS. 2-5.

The SEC elution profile for FIT-Ig6 shown in FIG. 7 revealed a major peak at the position expected for the six-polypeptide FIT-Ig6 monomer preceded by a barely detectable peak in the area of protein aggregates. The FIT-Ig6 binding protein was clearly obtained as a substantially completely homogenous product after one-step purification using Protein A affinity chromatography, without significant formation of aggregates. The percentage of aggregates is estimated at less than or equal to 0.1% (≤0.1%). FIT-Ig6 clearly surpassed all other FIT-Igs in having no significant percentage of aggregates.

Expression and SEC data for FIT-Igs 1-6 are shown in Table 7, below.

TABLE 7
Expression and SEC analysis of FIT-Ig binding proteins

| | Outer/Inner Fab | DNA molar ratio: | Expression | % Peak Monomeric |
|---|---|---|---|---|
| FIT-Ig protein | Specificity | Chain #1:#2:#3 | level (mg/L) | Fraction by SEC |
| FIT-Ig1 | PD-L1/EGFR | 1:3:3 | 6.66 | <30% |
| FIT-Ig2 | EGFR/PD-L1 | 1:3:3 | 5.30 | 74.7% |
| FIT-Ig3 | PD-L1/EGFR | 1:3:3 | 1.63 | 37.7% |
| FIT-Ig4 | EGFR/PD-L1 | 1:3:3 | 1.05 | 91.12% |
| FIT-Ig5 | PD-L1/EGFR | 1:3:3 | 8.24 | 98.8% |
| FIT-Ig6 | EGFR/PD-L1 | 1:3:3 | 11.00 | 99.9% |

The above data indicate:
FIT-Igs 1, 2, and 3 exhibit exceptionally high aggregate percentages and unacceptably low levels of expression in cultures of transfected HEK293 cells, and therefore cannot provide the quality (no or insignificantly low percentage of aggregates) and quantity of protein required for further pre-clinical assessment as therapeutic drugs
FIT-Ig4 has a lower aggregate percentage than FIT-Igs 1, 2, and 3, but the level is not insignificant for drug development. Moreover, FIT-Ig4 exhibits an unacceptably low level of expression. Therefore, FIT-Ig4 also cannot provide the quantity and quality of protein required for further pre-clinical assessments as a therapeutic drug
FIT-Ig5 has nearly acceptable levels of aggregates and expression, however, the level of expression of less than 10 mg/ml predicts that efforts required to isolate a stably transfected CHO cell line to obtained quantities for pre-clinical and clinical assessments will not be successful or cost-effective
FIT-Ig6 surprisingly exhibits both a high level of expression in cultures of transfected HEK293 cells and no significant amount of aggregate formation (≤0.1%). Thus, with respect to aggregate formation, FIT-Ig6 is more stable and has at least 10-fold lower percentage of aggregates than FIT-Ig5 after one-step purification using Protein A affinity chromatography. The levels of expression and exceptionally low level of aggregation of FIT-Ig6 expressed in mammalian cell cultures qualifies this binding protein for use as a candidate for both pre-clinical and clinical assessments as an anti-cancer therapeutic drug
The exceptional properties of FIT-Ig6 are the result of:
   1. using the anti-PD-L1 mAb 3G10 as the source of the VH and VL domains that form the PD-L1-specific antigen-binding site in each of the PD-L1 specific Fab binding units of FIT-Ig6,
   2. using the panitumumab anti-EGFR mAb as the source of the VH and VL domains that form the EGFR-specific antigen-binding site in each of the EGFR-specific Fab binding units of the FIT-Ig6,
   3. positioning the EGFR-specific Fab binding units as outer Fab binding units of FIT-Ig6, and
   4. positioning the PD-L1-specific Fab binding unit as the inner Fab binding units of FIT-Ig6

### Example 2: Binding Affinities of FIT-Ig5 and FIT-Ig6

Binding affinities of parental anti-EGFR panitumumab, parental anti-PD-L1 mAb 3G10, FIT-Ig5, and FIT-Ig6 were determined by biolayer interferometry. Briefly, each parental mAb and FIT-Ig was characterized for affinities and binding kinetics by Octet®RED96 biolayer interferometry (Pall FortéBio LLC). Each parental mAb and FIT-Ig were captured by Anti-Human IgG Fc Capture (AHC) Biosensors (Pall) at a concentration of 100 nM for 30 seconds. Sensors were then dipped into running buffer (1X, pH 7.2, PBS, 0.05% Tween 20, 0.1% BSA) for 60 seconds to check baseline. Binding was measured by dipping sensors into a single concentration of recombinant human PD-L1 (Novoprotein) or recombinant human EGFR (Sino Biological Inc) both ranging from 1 nM to 200 nM. Dissociation was followed by dipping sensors into running buffer for 1200 seconds. The association and dissociation curves were fitted to a 1:1 Langmuir binding model using ForteBio Data Analysis software (Pall). Results are shown in Table 8.

TABLE 8
Binding Affinities of Parental mAbs
and FIT-Igs for PD-L1 and EGFR

| Parental mAb | Target | kₒₙ | k_{off} | K_{D} |
|---|---|---|---|---|
| or FIT-Ig | Antigen | (M⁻¹ sec⁻¹) | (sec⁻¹) | (M) |
| panitumumab | EGFR | 3.55 × 10⁵ | 2.30 × 10⁻⁴ | 6.47 × 10⁻¹⁰ |
| FIT-Ig5 | | 1.62 × 10⁵ | 6.14 × 10⁻⁵ | 3.79 × 10⁻¹⁰ |
| FIT-Ig6 | | 2.18 × 10⁵ | 1.07 × 10⁻⁴ | 4.91 × 10⁻¹⁰ |
| 3G10 | PD-L1 | 9.79 × 10⁵ | 1.22 × 10⁻² | 1.25 × 10⁻⁸ |
| FIT-Ig5 | | 1.40 × 10⁶ | 1.56 × 10⁻² | 1.11 × 10⁻⁸ |
| FIT-Ig6 | | 8.86 × 10⁵ | 1.44 × 10⁻² | 1.63 × 10⁻⁸ |

The results indicate that the FIT-Ig5 and FIT-Ig6 possess binding affinities for the EGFR and PD-L1 target antigens that are similar to those of the parental panitumumab and parental mAb 3G10, respectively. In particular, the K_{D} of FIT-Ig6 for EGFR was approximately 25% lower than that of the parental panitumumab, and the K_{D} of FIT-Ig6 for PD-L1 was approximately 30% higher than that of the parental mAb 3G10. These levels of differences in K_{D} values of FIT-Ig6 and those of the parental mAbs were likely due to assay variations. Accordingly, FIT-Ig6 has substantially the same affinities as (i.e., same or within 30% of) the affinities for EGFR and PD-L1 as each of the parental antibodies from which the respective specificities were derived.

Accordingly, these data indicate that the FIT-Ig6 binding protein retained the binding affinities of the parental mAbs. Moreover, the binding affinities of the FIT-Ig6 binding protein are acceptable for continuing pre-clinical and clinical assessments of FIT-Ig6 as an anti-cancer drug.

### Example. 3. Pharmacokinetic Study of FIT-Ig6 in Male Sprague-Dawley Rats

Pharmacokinetic properties of FIT-Ig6 were assessed in male Sprague-Dawley (SD) rats. FIT-Ig protein was administered to male SD rats at a single intravenous dose of 5 mg/kg. Serum samples were collected at different time points over a period of 28 days with sampling at 0 minutes, 5 minutes, 15 minutes, 30 minutes, 1 hour, 2 hours, 4 hours, 8 hours, 24 hours, 2 days, 4 days, 7 days, 10 days, 14 days, 21 days, and 28 days serial bleeding via tail vein, and analyzed by general ELISAs. Briefly, ELISA plates were coated with 125 ng/well of goat anti-human IgG Fc antibody (Rockland, Cat#: 609-101-017) at 4° C. overnight, blocked with 1X PBS/1% BSA/0.05% Tween-20/0.05% ProClin™ 300. All serum samples were diluted 20-fold in blocking buffer first. Additional dilution was made in 5% pooled rat serum and incubated on the plate for 60 minutes at 37° C. Detection was carried out with anti-human IgG (Fab fragment) peroxidase conjugated (Sigma; catalogue no. A0293), and concentrations were determined with standard curves using the four-parameter logistic fit. Values for the pharmacokinetic parameters were determined by non-compartmental model using WinNonlin software (Pharsight Corporation, Mountain View, Calif.).

A graph of the serum concentration of the FIT-Ig6 versus time in three SD rats is shown FIG. 8.

An analysis of the results shown FIG. 8 for two of the animals (Rat #1 and Rat #3) yielded the PK parameters shown in Table 9, below. (The complete set of data for Rat #2 could not be analyzed with that of Rat #1 and Rat #3 owing to unresolved problems associated with the first two data points, which precluded analysis by software, although the remaining times points were in the normal range.)

TABLE 9
PK Parameters for FIT-Ig6 in Male Sprague-Dawley Rats

| PK parameters | Unit | Rat #1 | Rat #3 | Mean |
|---|---|---|---|---|
| CL | mL/day/kg | 7.52 | 6.02 | 6.77 |
| Vss | mL/kg | 101 | 105 | 103 |
| V1 | mL/kg | 58.0 | 56.0 | 57.0 |
| Alpha t_{1/2} | day | 0.148 | 0.189 | 0.168 |
| Beta t_{1/2} | day | 9.43 | 12.3 | 10.9 |
| AUC | day μg/mL | 665 | 830 | 747 |
| MRT | day | 13.4 | 17.5 | 15.5 |

CL (total clearance), Vss (volume of distribution at steady state), V1 (initial volume distribution), Alpha t_{1/2} (distribution half-life), Beta t_{1/2} (elimination half-life), AUC (area under the curve), MRT (mean residence time)

The above PK data indicate that FIT-Ig6 was stable in SD rats, and had similar PK parameters of conventional mAbs.

Importantly, the relatively long elimination half-life (Beta t_{1/2}=10.9 days) and low clearance (CL=6.77 mL/day/kg) of FIT-Ig6 will enable its therapeutic utility for chronic indications with less frequent dosing, similar to a therapeutic mAb.

The contents of all references (including literature references, patents, patent applications, and websites) that are cited throughout this application are hereby expressly incorporated by reference in their entirety. The practice of the present invention will employ, unless otherwise indicated, conventional techniques of immunology, molecular biology and cell biology, which are well known in the art.

The invention may be embodied in other specific forms without departing from the essential characteristics of the invention described above. The foregoing embodiments are therefore to be considered in all respects illustrative rather than limiting of the invention described herein. The scope of the invention is thus indicated by the appended claims rather than by the foregoing description, and all changes that come within the meaning and range of equivalency of the claims are therefore intended to be embraced herein.

1. A Fabs-In-Tandem Immunoglobulin (FIT-Ig) binding protein that binds EGFR and PD-L1 and that comprises a first polypeptide chain, a second polypeptide chain, and a third polypeptide chain, wherein:
the first polypeptide chain comprises, from the amino terminus to the carboxy terminus, VLEGFR-CL-VHPD-L1-CH1-Fc, wherein VLEGFR is an antibody light chain variable domain of a first parental antibody that binds EGFR, CL is an antibody light chain constant domain, VHPD L1 is an antibody heavy chain variable domain of a second parental antibody that binds PD-L1, CH1 is a first constant domain of an antibody heavy chain, Fc is an antibody Fc, wherein CL is fused directly to VHPD L1, wherein there is no artificial linker inserted between variable and constant domains, and wherein:
VLEGFR comprises amino acid residues 1-107 of SEQ ID NO:1 and
VHPD L1 comprises amino acid residues 215-331 of SEQ ID NO:1;
the second polypeptide chain comprises, from the amino terminus to carboxy terminus, VHEGFR-CH1, wherein VHEGFR, is an antibody heavy chain variable domain of said first parental antibody that binds EGFR, wherein CH1 is a first constant domain of an antibody heavy chain, wherein there is no artificial linker inserted between VHEGFR and CH1, and wherein:
VHEGFR comprises amino acid residues 1-119 of SEQ ID NO:2; and
the third polypeptide chain comprises, from the amino terminus to the carboxy terminus, VLPD-L1-CL, wherein VLPD-L1 is a light chain variable domain of said second parental antibody that binds PD-L1, wherein CL is an antibody light chain constant domain, wherein there is no artificial linker inserted between VLPD-L1 and CL, and wherein:
VLPD-L1 comprises amino acid residues 1-107 of SEQ ID NO:3.

2. The FIT-Ig binding protein according to claim 1, wherein the binding protein is a six-polypeptide chain binding protein comprising two of said first polypeptide chains, two of said second polypeptide chains, and two of said third polypeptide chains, wherein said polypeptide chains associate to form four Fab binding units, wherein two of the Fab binding units bind EGFR and two of the Fab binding units bind PD-L1.

3. (canceled)

4. The FIT-Ig binding protein according to claim 1, wherein said antibody CL domain in said first polypeptide chain and in said third polypeptide chain comprises amino acid residues 108-214 of SEQ ID NO:1.

5. (canceled)

6. The FIT-Ig binding protein according to claim 1, wherein said antibody CH1 domain present in said first polypeptide chain and in said second polypeptide chain comprises amino acid residues 332-434 of SEQ ID NO:1.

7. (canceled)

8. The FIT-Ig binding protein according to claim 1, wherein said antibody Fc present in said first polypeptide chain comprises amino acid residues 435-661 of SEQ ID NO:1.

9. The FIT-Ig binding protein according to claim 1, wherein:
said first polypeptide chain comprises an amino acid sequence according to SEQ ID NO:1;
said second polypeptide chain comprises an amino acid sequence according to SEQ ID NO:2; and
said third polypeptide chain comprises an amino acid sequence according to SEQ ID NO:3.

10. (canceled)

11. (canceled)

12. A composition comprising the FIT-Ig binding protein according to claim 1, wherein said composition comprises less than or equal to 0.1% FIT-Ig binding protein aggregates.

13. (canceled)

14. (canceled)

15. (canceled)

16. (canceled)

17. (canceled)

18. (canceled)

19. (canceled)

20. (canceled)

21. (canceled)

22. An isolated nucleic acid molecule encoding one or more of:
a first polypeptide chain that comprises an amino acid sequence according to SEQ ID NO:1;
a second polypeptide chain comprises an amino acid sequence according to SEQ ID NO:2; and
a third polypeptide chain comprises an amino acid sequence according to SEQ ID NO:3.

23. (canceled)

24. A vector comprising one or more isolated nucleic acid molecules according to claim 22, wherein said vector is an expression vector and said one or more isolated nucleic acids is operably linked to transcriptional and translational sequences that permit expression of the encoded one or more polypeptide chains.

25. The vector according to claim 24 selected from the group consisting of: pcDNA, pcDNA3.1, pTT, pTT3, pEFBOS, pBV, pJV, pcDNA3.1 TOPO, pEF6 TOPO, and pBJ.

26. (canceled)

27. An isolated host cell comprising one or more expression vectors, wherein said one or more vectors encodes the three polypeptide chains that form an EGFR/PD-L1 FIT-Ig binding protein according to claim 1.

28. The isolated host cell according to claim 27, wherein said host cell is an isolated prokaryotic host cell.

29. The isolated host cell according to claim 27, wherein said host cell is an isolated eukaryotic host cell.

30. (canceled)

31. The isolated mammalian host cell according to claim 30, wherein said eukaryotic host cell is an isolated mammalian host cell is selected from the group consisting of: a Chinese Hamster Ovary (CHO) cell, a COS cell, a Vero cell, an SP2/0 cell, an NS/0 myeloma cell, a human embryonic kidney (HEK293) cell, a baby hamster kidney (BHK) cell, a HeLa cell, a human B cell, a CV-1/EBNA cell, an L cell, a 3T3 cell, an HEPG2 cell, a PerC6 cell, and an MDCK cell.

32. A method of producing an EGFR/PD-L1 FIT-Ig binding protein, comprising culturing an isolated mammalian host cell according to claim 30 under conditions sufficient to produce an EGFR/PD-L1 FIT-Ig binding protein.

33. The method according to claim 32, wherein said mammalian host cell is a HEK293 cell.

34. The method according to claim 33, wherein said FIT-Ig binding protein is expressed at a level of greater than 10 mg/L.

35. (canceled)

36. (canceled)

37. (canceled)

38. A method of treating cancer in a human subject comprising administering to the subject an EGFR/PD-L1 FIT-Ig binding protein according to claim 1.

39. (canceled)

40. The method according to claim 38, wherein said cancer is selected from the group consisting of: a melanoma, a renal cancer, a prostate cancer, a pancreatic adenocarcinoma, a breast cancer, a colon cancer, a lung cancer, an esophageal cancer, a squamous cell carcinoma of the head and neck, a liver cancer, an ovarian cancer, a cervical cancer, a thyroid cancer, a glioblastoma, a glioma, a leukemia, and a lymphoma.

41. The method according to claim 40, wherein said melanoma is a metastatic malignant melanoma, said renal cancer is a clear cell renal cell carcinoma, said prostate cancer is a hormone refractory prostate adenocarcinoma, or said lung cancer is a non-small cell lung cancer.

42. (canceled)

43. (canceled)

44. (canceled)

45. (canceled)

46. (canceled)

47. (canceled)

48. (canceled)

49. (canceled)

50. (canceled)

51. (canceled)
